(19) United States
(12) Patent Application Publication
(10) Pub. No.: US 2025/0288726 A1
(43) Pub. Date: Sep. 18, 2025

(54) SYSTEMS AND METHODS FOR SHEAR-THINNING HEMOSTATS, INCLUDING THERMORESPONSIVE HEMOSTATS

(71) Applicant: Terasaki Institute for Biomedical Innovation, Los Angeles, CA (US)

(72) Inventors: Marvin M. Mecwan, Los Angeles, CA (US); Alireza Khademhosseini, Los Angeles, CA (US); Johnson Vitharikunnil John, Los Angeles, CA (US)

(73) Assignee: Terasaki Institute for Biomedical Innovation, Los Angeles, CA (US)

(21) Appl. No.: 18/868,963

(22) PCT Filed: Jun. 1, 2023
(86) PCT No.: PCT/US2023/024125

§ 371 Date: Nov. 25, 2024

Related U.S. Application Data
(60) Provisional application No. 63/348,119, filed on Jun. 2, 2022.

(51) Int. Cl. A61L 27/44; A61L 27/52; A61L 27/54

(52) U.S. Cl. CPC A61L 27/446; A61L 27/52; A61L 27/54; A61L 2400/04; A61L 2430/36

(57) ABSTRACT

The present disclosure generally relates to methods and systems for shear-thinning hemostats, for example, to use on a site of hemorrhage. In some embodiments, the hemostat is a solution comprising a polymer and a plurality of nanoparticles, where the solution is thixotropic, shear-thinning, and/or comprises a lower critical solution temperature. In some embodiments, the injectable hemostat is a liquid at 25° C. and a solid at 37° C. In some embodiments, the disclosure relates to injecting a solution into a site of hemorrhage in a subject, where upon injecting the solution, a plug is formed, thus limiting blood loss in the subject. In some embodiments, injecting the solution within the site of hemorrhage reduces the time for the plug to form, relative to sites not treated with the liquid solution.

### RELATED APPLICATIONS

This application claims the benefit of U.S. Provisional Patent Application Ser. No. 63/348,119, filed Jun. 2, 2022, entitled “Systems and Methods for Shear-Thinning Hemostats,” by Mecwan, et al., incorporated herein by reference in its entirety.

### GOVERNMENT FUNDING

This invention was made with government support under Grant No. HL137193 awarded by National Institutes of Health. The government has certain rights in the invention.

### FIELD

The current disclosure generally relates to systems and methods for shear-thinning hemostats.

### BACKGROUND

Hemorrhage is a major cause of morbidity and mortality following traumatic injury in both the military and civilian sectors. For example, hemorrhage is common in subjects experiencing extremity wounds, e.g., a lost limb, as well as in subjects exposed to blunt trauma due to artillery blasts, car accidents, etc. Importantly, it has been estimated that hemorrhage is one of the most preventable forms of death on the battlefield. However, current technologies do not permit facile treatment of such complex injuries by personnel with basic medical training in a prehospital setting and in austere environments with limited resources. Therefore, improvements are needed.

### SUMMARY

The present disclosure generally relates to systems and methods for shear-thinning hemostats. For example, in some embodiments, a solution comprising a thermoresponsive polymer and a plurality of nanoparticles capable of forming a plug within a site of hemorrhage is provided. The subject matter of the present disclosure involves, in some cases, interrelated products, alternative solutions to a particular problem, and/or a plurality of different uses of one or more systems and/or articles.

In some embodiments, the present disclosure relates to an article, comprising a thermoresponsive polymer dissolved in a solution and a plurality of nanoparticles suspended in the solution, wherein the solution is thixotropic and comprises a lower critical solution temperature (LCST) of at least 25° C.

In some embodiments, the disclosure teaches an article, comprising a thermoresponsive polymer dissolved in a thixotropic solution, wherein the polymer is insoluble in the solution at between 32° C. and 37° C., and a plurality of charged particles suspended in the thixotropic solution.

Other aspects of the disclosure relate to a method comprising injecting a liquid solution into a site of hemorrhage in a subject, wherein the liquid solution comprises a polymer and a plurality of nanoparticles, wherein upon injecting the liquid solution the polymer produces a hydrogel and the plurality of nanoparticles concentrate one or more clotting factors at the site of hemorrhage, and forming a plug within the site of hemorrhage.

In certain embodiments, the disclosure teaches a method comprising administering, to a site of hemorrhage in a subject, a solution comprising a polymer and charged silicate nanoparticles, wherein upon administration, the solution is heated by blood at the site of hemorrhage to produce a hydrogel comprising the polymer and clotting proteins inter-dispersed with the charged silicate nanoparticles.

In certain aspects, the current disclosure further relates to a thixotropic solution comprising a polymer able to form a hydrogel at a temperature between 32° C. and 37° C., and charged lithium sodium magnesium silicate particles suspended in the thixotropic solution.

In some embodiments, the disclosure also teaches an article comprising a solution comprising a polymer able to form a hydrogel at a temperature between 32° C. and 37° C., and charged lithium sodium magnesium silicate particles.

Additional embodiments of the disclosure relate to an article comprising a solution comprising poly(N-vinyl caprolactam) and charged lithium sodium magnesium silicate particles.

In addition, certain aspects of the disclosure relate to a method comprising administering, to a site of hemorrhage in a subject, a solution comprising a polymer and charged silicate nanoparticles, and heating the solution at the site of hemorrhage to the subject's temperature to produce a hydrogel comprising the polymer and clotting proteins inter-dispersed with the charged silicate nanoparticles.

In another aspect, the present disclosure encompasses methods of making one or more of the embodiments described herein, for example, shear-thinning hemostats. In still another aspect, the present disclosure encompasses methods of using one or more of the embodiments described herein, for example, shear thinning hemostats.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 shows the preparation of an injectable hemostat for hemorrhage control and antibiotic drug delivery, according to some embodiments;

FIGS. 2A-C show photomicrographs of an injectable hemostat at 25° C. (FIG. 2A) and 37° C. (FIG. 2B), while FIG. 2C shows representative scanning electron micrographs of the injectable hemostat, according to some embodiments;

FIGS. 3A-F show the shear thinning properties of an injectable hemostat at 25° C. and 37° C., respectively, including shear stress versus shear rate (FIG. 3A and FIG. 3D), viscosity versus shear rate (FIG. 3B and FIG. 3E), and storage modulus versus time (FIG. 3C and FIG. 3F), according to some embodiments;

FIGS. 4A-4C show the injection force measurement setup (FIG. 4A) and injection force curves (FIG. 4B and FIG. 4C) for injectable hemostats with varying compositions, according to some embodiments;

FIGS. 5A-5D show the effect of the injectable hemostat on the clotting time of blood;

FIGS. 6A-6D show the experimental setup and quantification of the efficacy of an injectable hemostat at minimizing blood loss in an ex vivo hemorrhage model, according to some embodiments;

FIGS. 7A-7B show the cumulative drug release from doxycycline-loaded injectable hemostats, according to some embodiments;

FIGS. 8A-8C show the zones of inhibition of doxycycline-loaded injectable hemostats, according to some embodiments;

FIGS. 9A-9B show amplitude sweeps of solutions comprising polyNIPAM and Laponite nanoparticles at 25° C. (FIG. 9A) and 37° C. (FIG. 9B), according to some embodiments;

FIGS. 10A-10B show frequency sweeps of polyNIPAM and Laponite shear-thinning hydrogels acquired at 0.1% strain at 25° C. (FIG. 10A) and 37° C. (FIG. 10B), according to some embodiments;

FIG. 11 shows the viscosity of solutions comprising polyNIPAM and Laponite nanoparticles at room temperature (25° C.) and body temperature (37° C.), according to some embodiments;

FIGS. 12A-12B show the temperature-dependent change in G′ and G″ values of solutions comprising polyNIPAM and Laponite nanoparticles as the temperature slowly ramps up from 15° C. to 45° C. (FIG. 12A), and as the temperature is suddenly changed from 25° C. to 37° C. (FIG. 12B), according to some embodiments;

FIGS. 13A-13B show the cell viability of NIH/3T3 mouse fibroblasts following 5 days of coculture with various shear-thinning hydrogels comprising polyNIPAM and Laponite nanoparticles using PrestoBlue Cell Viability Reagent (FIG. 13A) and a LIVE/DEAD Assay (FIG. 13B), according to some embodiments;

FIG. 14 shows a plot of the hemolysis ratio of various shear-thinning hydrogels comprising of polyNIPAM and Laponite nanoparticles; according to some embodiments; and

FIGS. 15A and 15B show a plot of the mass loss of shear-thinning hydrogels comprising polyNIPAM and Laponite nanoparticles following incubation at 37° C. in phosphate-buffered saline for 48 hours (FIG. 15A) and human plasma (FIG. 15B), respectively; according to some embodiments;

FIG. 16A-16D shows data from an in vivo rat liver bleeding model using articles disclosed herein. FIG. 16A is a schematic of the in vivo bleeding model prepared using BioRender. FIG. 16B shows images of blood loss (captured on Whatman filter paper) following injury to the liver and treatment with either controls (e.g., sham or Floseal) or articles of the present invention (e.g., 10N3L. FIGS. 16C and 16D show plots of the mass of blood loss (determined via weight of blood-soaked Whatman filter paper) and clotting times, respectively; according to some embodiments; and

FIG. 17 shows digital images of the hemocompatibility of the articles of the present invention compared to 2% SDS and saline, according to some embodiments;

FIG. 18A-18D shows scanning electron micrographs of platelet adhesion to p(NIPAM) and Laponite based T-STH. Low magnification images of 5N3L (FIG. 18A) and 10N3L (FIG. 18C) and higher magnification images of 5N3L (FIG. 18B) and 10N3L (FIG. 18D), according to some embodiment;

FIG. 19 shows a series of time-lapse digital images of T-STH gels being washed away from an injured liver using cold saline without rebleeding and without leaving any residue, according to some embodiments.

### DETAILED DESCRIPTION

The present disclosure generally relates to methods and systems for shear-thinning hemostats, for example, to use on a site of hemorrhage. In some embodiments, the hemostat is a solution comprising a polymer and a plurality of nanoparticles, where the solution is thixotropic, shear-thinning, and/or comprises a lower critical solution temperature (LCST). In some embodiments, the injectable hemostat is a liquid at 25° C. and a solid at 37° C. In some embodiments, the disclosure relates to injecting a solution into a site of hemorrhage in a subject, where upon injecting the solution, a plug is formed, thus limiting blood loss in the subject. In some embodiments, injecting the solution within the site of hemorrhage reduces the time for the plug to form, relative to sites not treated with the liquid solution.

In some cases, a hemostat may include a solution comprising a thermo-responsive polymer and a plurality of charged nanoparticles. In some embodiments, the solution may be injected into a site of hemorrhage in a subject, where the subjects body temperature (or other heat source) turns the solution into a solid plug. In some cases, the plurality of nanoparticles may stimulate coagulation within the site of hemorrhage, which may help stop bleeding with a blood clot (e.g., a fibrin-based hydrogel). In other embodiments, the nanoparticles may concentrate clotting factors within the site of hemorrhage (e.g., by binding them to their charged surfaces), which may accelerate the rate of plug formation (e.g., hydrogel formation and/or blood clotting), e.g., in addition to increasing the plug strength.

In some embodiments, a plug, comprising a polymer, formed within a site of hemorrhage may be washed away, for example, after a subject has reached a tertiary care unit or other medical care. In some cases, the plug may be dissolved by washing the site of hemorrhage with a physiological solution (e.g., 0.9% w/v saline), e.g., cooled to a temperature below the solution's lower critical solution temperature (LCST). In some cases, the plug may be broken apart and removed, for example, during debridement of the site of hemorrhage. In some cases, the plug may be left within the site of hemorrhage and allowed to degrade over time.

In some embodiments, a shear-thinning hemostat may comprise a polymer dissolved in a solution. In some cases, the polymer may include a thermo-responsive polymer and the solution may comprise a lower critical solution temperature (LCST), e.g., between 25° C. and 37° C. In some embodiments, the thermo-responsive polymer may comprise a polymer such as a poly(acrylamide), a poly(caprolactam), and/or a poly(vinyl ether), etc. The polymer, in certain cases, may comprise a copolymer, branched polymer, block copolymer, tri-block copolymer, etc. In some embodiments, at least one segment (or block) comprises a thermo-responsive polymer. In some embodiments, the solution comprising the polymer is a liquid at about 25° C. (i.e., room temperature) so that the solution may be injected, for example, into a site of hemorrhage. In some embodiments, the solution comprising the polymer is a solid at between 32° C. to 37° C., e.g., so that the solution forms a solid plug, for example, following injection into a subject (body temperature is approximately 37° C.), thus minimizing blood loss from a site of hemorrhage.

In certain embodiments, a shear-thinning hemostat may comprise a solution comprising a plurality of nanoparticles. In some embodiments, the plurality of nanoparticles comprises a charged outer surface, e.g., a negative or positively charged surface. The nanoparticles may comprise any type of nanoparticle (e.g., polymer, metal, ceramic, carbon, etc.). In some cases, the plurality of nanoparticles may be present in a solution, e.g., as discussed herein. In certain instances, the nanoparticles are uniformly and stably dispersed within the solution (e.g., they do not form aggregates). In some cases, the solution may be injected into a site of hemorrhage, e.g., such that after injection, the solution may form a solid plug within the site of hemorrhage. In certain cases, the plurality of nanoparticles is dispersed throughout the plug at formation and the nanoparticles may bind to, or adsorb, one or more blood products (e.g., clotting factors, platelets, etc.), thus concentrating them within the plug. In some embodiments, concentrating the blood products within the plug may accelerate coagulation and/or enhance certain mechanical properties of the plug (e.g., its viscoelastic properties).

Some aspects of the current disclosure relate to an injectable solution comprising a thermoresponsive polymer and a plurality of nanoparticles. In some embodiments, the injectable solution may be in a liquid state or a gel state (i.e., more viscous than the liquid state but less viscous than a solid state), for example, when the solution is below a lower critical solution temperature (LCST). In some embodiments, the state of the solution may depend, for example, on the concentration of polymer and/or the plurality of charged nanoparticles in the solution. In certain embodiments, the solution is always in the gel state (i.e., the storage modulus, G′, is greater than the loss modulus, G″), regardless of the concentration of the polymer and/or charged nanoparticle solution. In some embodiments, the solution (e.g., liquid and/or gel) is thick and/or viscous under static conditions, but may flow (i.e., become less viscous) when shaken, agitated, shear-stressed, or otherwise stressed (i.e., the solution is thixotropic and/or shear-thinning). This may facilitate delivery of the solution using a syringe, e.g., a hypodermic syringe.

In some embodiments, the injectable solution may have a first viscosity (e.g., between 5 Pas and 80 Pa s) at a temperature below the LCST of the solution (e.g., 32° C. for NIPAm-based solutions, or when in the liquid state) and a second viscosity (e.g., 10 and 400 Pa s) at a temperature above the LCST of the solution (e.g., 37° C. for NIPAm-based solutions, or when in the solid state). In certain embodiments, the solution may have a first storage modulus (e.g., between 5 Pa and 30 Pa) at a temperature below the LCST of the solution (e.g., 25° C. for NIPAm-based solutions, or when in the liquid state) and a second storage modulus (e.g., 0.5 and 500 Pa) at a temperature above the LCST of the solution (e.g., 37° C. for NIPAm-based solutions, or when in the solid state).

In some embodiments, an injectable hemostatic solution comprising a thermoresponsive polymer and a plurality of nanoparticles may exhibit a shear-thinning behavior (i.e., the viscosity decreases under shear strain). For example, in some cases, the solution may exhibit a decrease in viscosity when exposed to a first range of shear rates (e.g., between 1 s⁻¹ and 10 s⁻¹) at a temperature below the LCST of the solution (e.g., 25° C. for NIPAm-based solutions, or when in the liquid state); in other cases the solution may exhibit a decrease in viscosity when exposed to a second range of shear rates (e.g., between 0.01 s⁻¹ and 10 s⁻¹) at a temperature above the LCST of the solution (e.g., 37° C. for NIPAm-based solutions, or when in the solid state).

In certain embodiments, a solution may be thixotropic, that is, the solution may require time, following removal of an externally applied strain (e.g., an oscillatory strain), to return to an initial storage modulus (i.e., original storage modulus). For example, in some embodiments a solution may require a first time (e.g., between 1 sec and 60 sec), following removal of the external strain, to return to at least a percentage (e.g., between 90% and 100%) of the first initial storage modulus at a temperature below the LCST of the solution (e.g., 25° C. for NIPAm-based solutions, or when in the liquid state). In some embodiments, a solution may require a second time (e.g., between 1 sec and 60 sec), following removal of the external strain, to return to at least a percentage (e.g., between 90% and 100%) of the second initial 30 storage modulus at a temperature above the LCST of the solution (e.g., 37° C. for NIPAm-based solutions, or when in the solid state).

In some embodiments, the injectable solution may have a first storage modulus (G′) and a first loss modulus (G″) following exposure to a range of strains. In some cases, the solution may exhibit a G′ of between 20 Pa and 200 Pa and a G″ of between 5 Pa and 20 Pa when exposed to a first strain range of between 0% and 10% of axial strain at a temperature below the LCST of the solution (e.g., 25° C. for NIPAm-based solutions, or when in the liquid state). In certain embodiments, the solution may exhibit a G′ of between 50 Pa and 300 Pa and a G″ of between 10 Pa and 40 Pa when exposed to a second range of strain between 0% and 10% of axial strain at a temperature above the LCST of the solution (e.g., 37° C. for NIPAm-based solutions, or when in the solid state).

In some embodiments, the injectable solution may have a first and second storage modulus (G′) and a first and second loss modulus (G″) following exposure to a range of angular frequencies. In some cases, the solution may exhibit a first G′ of between 40 Pa and 100 Pa and a first G″ of between 5 Pa and 20 Pa when exposed to a first angular frequency range of between 0.1 and 100 rad/s at a temperature below the LCST of the solution (e.g., 25° C. for NIPAm-based solutions, or when in the liquid state). In certain embodiments, the solution may exhibit a second G′ of between 20 Pa and 200 Pa and a second G″ of between 4 Pa and 100 Pa when exposed to a first angular frequency range of between 0.1 and 100 rad/s at a temperature above the LCST of the solution (e.g., 37° C. for NIPAm-based solutions, or when in the solid state).

In some embodiments, the injectable solution may have a first and second storage modulus (G′) and a first and second loss modulus (G″) following exposure to a first range of temperatures and a second range of temperatures, respectively. In some cases, the solution may exhibit a G′ of between 10 Pa and 100 Pa and a G″ of between 8 Pa and 15 Pa when exposed to a first temperature range of between 0° C. and 30° C. In certain embodiments, the solution may exhibit a second G′ of between 10 Pa and 3000 Pa and a G″ of between 8 Pa and 500 Pa when exposed to a second temperature range of between 30° C. and 50° C.

In some embodiments, the injectable solution may be at a first temperature (e.g., 25° C.) prior to use and at a second temperature (e.g., 37° C.) after use (i.e., following injection into a site of hemorrhage). In some embodiments, the injectable solution may take between 10 sec and 30 sec to begin gelation, as determined by an increase in the G′ value. In other embodiments, the injectable solution may reach maximal G′ (e.g., between 1500 Pa and 3000 Pa) within between 50 sec and 90 sec of initiation of the temperature change.

In some cases, an injectable solution may be delivered to a site of hemorrhage, for example, using a syringe-based delivery device. In some embodiments, the hemostatic solution may be a viscous liquid or gel and exhibit shear-thinning behavior at a temperature between 25° C. and 37° C. In some embodiments, extrusion of the solution through a barrel of a needle may expose the hemostatic solution to a high shear force, thus reducing the viscosity of the fluid and the overall force that must be applied to the syringe-based delivery device. For example, in some cases, the injection force needed to deliver the hemostatic solution through a syringe comprising a needle (e.g., 23G needle) may be between 5 N and 15 N. In some embodiments, the injection force is greater than or equal to 5 N, greater than or equal to 10 N, greater than or equal to 15 N, etc. In other embodiments, the injection force is less than or equal to 15 N, less than or equal to 10 N, less than or equal to 5 N, etc.

In some embodiments, an injectable solution may comprise one or more therapeutic agents. In some cases, the therapeutic agent comprises one or more antibiotic compounds. Examples of antibiotic compounds include, but are not limited to, penicillin, macrolides, cephalosporins, fluoroquinolones, beta-lactams, etc. More than one may be used in some cases. In certain embodiments, the solution comprises one or more recombinant clotting factors, such as, for example, thrombin, factor XIII, and factor VII. In other cases, the solution comprises one or more antifibrinolytic agents, e.g., tranexamic acid and epsilon-aminocaproic acid. Combinations are also possible (e.g., the solution may comprise an antibiotic, clotting factor, and an antifibrinolytic agent etc.).

In some cases, an injectable solution comprising a thermoresponsive polymer, a plurality of particles, and a therapeutic agent may exist as a liquid at a first temperature below the LCST of the solution (e.g., 25° C.). In certain cases, the solution may exist as a solid hydrogel (or plug) at a second temperature above the LCST of the solution (e.g., 37° C.). In some embodiments, the therapeutic agent may diffuse out of the solid hydrogel over a period of hours to days to weeks. For example, in some embodiments, the cumulative release of the therapeutic agent from the hydrogel is between 0 and 100% (by weight) over a period of about 30 days. For example, in some embodiments, the cumulative release of the therapeutic agent is greater than or equal to 0%, greater than or equal to 20%, greater than or equal to 40%, greater than or equal to 60%, greater than or equal to 80%, greater than or equal to 100%, etc., after 30 days. In addition, in some embodiments, the cumulative release of the therapeutic agent is less than or equal to 100%, less than or equal to 80%, less than or equal to 60%, less than or equal to 40%, less than or equal to 20%, less than or equal to 0%, etc., after 30 days. Combinations of any of these ranges are also possible.

Certain aspects of the disclosure relate to methods of treating a site of hemorrhage using an injectable solution comprising a thermo-responsive polymer dissolved in a solution and a plurality of charged nanoparticles suspended in the solution. In some embodiments, the polymer is a thermoresponsive polymer. In some cases, a surface of the plurality of nanoparticles has either a positive charge (i.e., a positive zeta potential) or a negative charge (i.e., a negative zeta potential). In some cases, the solution is a liquid at a temperature of about 25° C. to about 32° C. The solution may form a hydrogel at a temperature of about 32° C. to about 37° C. in certain embodiments. In some embodiments, the solution can be used as an injectable hemostat, for example, to plug a wound at a site of hemorrhage in a subject in need thereof.

Those of ordinary skill will appreciate that the current standard for the treatment of a subject experiencing a compressible traumatic injury (e.g., a traumatic amputation that can be easily assessed and externally compressed without cutting into the body) is primarily focused on hemorrhage control followed by reversing hypovolemia to prevent co-morbidities such as cardiac arrest. Traditionally, hemorrhage control has been achieved by packing the injury with gauze (e.g. QuikClot Combat Gauze) which serves to absorb the blood and to stimulate native blood clot formation, resulting in a plug at the site of hemorrhage. In many instances, these products are embedded with various clotting factors configured to stimulate native clot formation. However, the clots formed (i.e., plugs) are mechanically weak and are not able to maintain hemorrhage control, for example, during prolonged evacuations in austere (or remote) environments.

One set of embodiments of the current disclosure is generally directed to an injectable hemostatic solution that forms a multifunctional plug following injection into a site of hemorrhage. The solution may comprise a thermoresponsive polymer and a plurality of nanoparticles. The multifunctional plug may be useful in establishing hemorrhage control in a subject. For example, in some embodiments, the multifunctional plug comprises a hydrogel component that can physically obstruct blood flow out of a wound (e.g., a site of hemorrhage), thus allowing innate coagulation process to proceed and a native blood clot to form at the site of hemorrhage. In some cases, the subject's own body temperature (e.g., which may be between 32° C. and 37° C.) may facilitate formation of a hydrogel. In addition, in certain cases, an external heat source (e.g., a heat gun) may be used to facilitate formation of the hydrogel. In some embodiments, the heat may cause the thermo-responsive polymer to undergo a phase change to form a hydrogel.

In some embodiments, a multifunctional plug comprises a hydrogel comprising a polymeric network that can swell and/or absorb one or more blood components (e.g., proteins, cells, fluids) within the polymeric network. This may, in certain cases, reduce the total volumetric blood loss at a site of hemorrhage. In some cases, the hydrogel may have a swelling ratio (defined as the fractional increase in the weight of the hydrogel due to water absorption) of between 1 and 100. For example, the swelling ratio of the hydrogel may be greater than or equal to 1, greater than or equal to 3, greater than or equal to 10, greater than or equal to 30, greater than or equal to 50, greater than or equal to 75, greater than or equal to 100, etc., according to some embodiments. In other cases, the hydrogels swelling ratio may be less than or equal to 100, less than or equal to 75, less than or equal to 50, less than or equal to 30, less than or equal to 10, less than or equal to 3, less than or equal to 1, etc. Combinations of any of these ranges are also possible. For instance, the swelling ratio may be between 10 and 100, between 50 and 75, between 1 and 10, etc.

In another set of embodiments, a multifunctional plug may comprise a blood clot. In some cases, the addition of a solution comprising a thermoresponsive polymer and a plurality of nanoparticles to a site of hemorrhage, forms a hydrogel plug within the site of hemorrhage and slows blood loss at the site of hemorrhage. In some situations, slowing the blood loss may result in the formation of a native blood clot at the site of hemorrhage. In some embodiments, the plurality of nanoparticles within the hydrogel may bind to blood cells (e.g., platelets) and/or blood proteins (e.g., clotting factors). In some cases, the nanoparticles may stimulate (e.g., by activating the clotting cascade) and/or accelerate (e.g., by increasing the local concentration of clotting factors and blood platelets) blood clot formation within the site of hemorrhage.

Without wishing to be bound by any theory, it is believed that since many hydrogels are porous, blood proteins and blood cells may penetrate and concentrate inside the hydrogel, such that the resultant blood clot (e.g., composed primarily of a fibrin hydrogel) and the polymer hydrogel become entangled, thus ensuring the hydrogel (and hence the nanoparticles) remains within the site of hemorrhage (for instance, it may be more difficult to wash away or dilute the hydrogel at the site of hemorrhage). Such configurations, according to some embodiments, may transform a subject's blood (which is often abundant at a site of hemorrhage) into a plug or an “elastic bandage.” This may be helpful in minimizing materials, equipment, personnel, etc., needed to treat a hemorrhaging subject, at least in certain cases.

Thus, in some embodiments, the charged nanoparticles may be useful to transform a site of hemorrhage into a blood-based bandage. This may be surprising since most nanoparticles are generally regarded as toxic. For example, intravenous delivery of charged nanoparticles is known by those of ordinary skill in the art to cause a depletion of coagulation factors and blood platelets through formation of small intravascular clots leading to a condition known as disseminated intravascular coagulation (or consumptive coagulopathy), which left untreated may cause organ failure and death. Accordingly, it is surprising that nanoparticles can be used to stop bleeding at a site of hemorrhage.

In some embodiments, injecting a solution comprising a thermoresponsive polymer and a plurality of nanoparticles within a site of hemorrhage may reduce the time for a plug to form and/or bleeding to stop, relative to sites of hemorrhages treated with a gauze-based product. For example, in some cases, injecting the solution within a site of hemorrhage reduces the time for a plug to form by greater than or equal to 30%, greater than or equal to 40%, greater than or equal to 50%, greater than or equal to 60%, greater than or equal to 70%, greater than or equal to 80%, greater than or equal to 90%, etc., relative to sites of hemorrhages treated with a gauze-based product. In other cases, injecting the solution within a site of hemorrhage reduces the time for a plug to form by less than or equal to 90%, less than or equal to 80%, less than or equal to 70%, less than or equal to 60%, less than or equal to 50%, less than or equal to 40%, etc., relative to sites of hemorrhages treated with a gauze-based product. Combinations of these are also possible, e.g., between 30% and 90%, between 50% and 80%, etc.

In other embodiments, injecting a solution comprising a thermoresponsive polymer and a plurality of nanoparticles within a site of hemorrhage may increase the firmness of a plug formed by between 10% to 50, relative to sites of hemorrhages treated with a gauze-based product. For example, in some cases, injecting the solution within a site of hemorrhage increases the firmness of the plug by greater than or equal to 10%, greater than or equal to 20%, greater than or equal to 30%, greater than or equal to 40%, greater than or equal to 50%, etc., relative to sites of hemorrhages treated with the gauze-based product. In other cases, injecting the solution within a site of hemorrhage reduces the time for a plug to form by less than or equal to 50%, less than or equal to 40%, less than or equal to 30%, less than or equal to 20%, less than or equal to 10%, etc., relative to sites of hemorrhages treated with the gauze-based product.

In some embodiments, injecting a solution comprising a thermoresponsive polymer and a plurality of nanoparticles within a site of hemorrhage increases the maximum strength of a plug by between 10% to 50%, relative to sites of hemorrhages treated with a gauze-based product (e.g., Quikclot Combat Gauze). For example, in some cases, injecting the solution within a site of hemorrhage increases the maximum strength of the plug by greater than or equal to 10%, greater than or equal to 20%, greater than or equal to 30%, greater than or equal to 40%, greater than or equal to 50%, etc., relative to sites of hemorrhages treated with the gauze-based product. In other cases, injecting the solution within a site of hemorrhage increases the maximum strength of the plug by less than or equal to 50%, less than or equal to 40%, less than or equal to 30%, less than or equal to 20%, less than or equal to 10%, etc., relative to sites of hemorrhages treated with the gauze-based product.

In some embodiments, injecting a solution comprising a thermoresponsive polymer and a plurality of nanoparticles within a site of hemorrhage decreases the percent lysis at 30 minutes (LY30) of the plug by between 10% to 50%, relative to sites of hemorrhages treated with a gauze-based product. For example, in some cases, injecting the solution within a site of hemorrhage decreases the LY30 of the plug by greater than or equal to 10%, greater than or equal to 20%, greater than or equal to 30%, greater than or equal to 40%, greater than or equal to 50%, etc., relative to sites of hemorrhages treated with the gauze-based product. In other cases, injecting the solution within a site of hemorrhage decreases the LY30 of the plug by less than or equal to 50%, less than or equal to 40%, less than or equal to 30%, less than or equal to 20%, less than or equal to 10%, etc., relative to sites of hemorrhages treated with the gauze-based product.

In some embodiments, injecting a solution comprising a thermoresponsive polymer and a plurality of nanoparticles within a site of hemorrhage slows the rate of blood loss from a site of hemorrhage to between 1 mL/hr and 10 mL/hr, relative to sites of hemorrhages treated with a gauze-based product. For example, in some cases, injecting the solution within a site of hemorrhage slows the rate of blood loss from a site of hemorrhage by greater than or equal to 1 mL/hr, greater than or equal to 2 mL/hr, greater than or equal to 4 mL/hr, greater than or equal to 8 mL/hr, greater than or equal to 10 mL/hr, etc., relative to sites of hemorrhages treated with a gauze-based product. In other cases, injecting the solution within a site of hemorrhage slows the rate of blood loss from the site of hemorrhage by less than or equal to 10 mL/hr, less than or equal to 8 mL/hr, less than or equal to 4 mL/hr, less than or equal to 2 mL/hr, less than or equal to 1 mL/hr, etc., relative to sites of hemorrhages treated with a gauze-based product.

In some instances, a multifunctional hemostatic plug, formed from injecting a solution comprising a thermoresponsive polymer and a plurality of nanoparticles, may need to be removed from a subject, for example, after transport to a higher echelon of care. In some embodiments, the hemostatic plug may be removed by washing the site of hemorrhage with a physiologic solution (e.g., Plasma-Lyte A, dextran 70, saline 0.9%, glucose, etc.), which in some cases may be cooled to a temperature below the lower critical solution temperature (LCST) of the starting solution. In some embodiments, the LCST of the solution is between 32° C. and 37° C. In certain cases, the plug may be broken apart and removed, for example, during debridement of the site of hemorrhage. In some embodiments, the plug may be left within the site of hemorrhage and allowed to degrade, for instance, into metabolites that may be readily processed by the body. Combinations are also possible, for example, a surgeon may wash the site of hemorrhage with a cooled solution (e.g., below the LCST of the solution) while debriding the wound bed.

In some cases, administering a solution comprising a thermoresponsive polymer and a plurality of nanoparticles to a site of hemorrhage produces a multifunctional hemostatic plug. The plug may be helpful to prevent or slow blood loss at the site of hemorrhage in a subject. In some embodiments, the solution may be administered to an external compressible wound (e.g., an amputated limb). In some cases, the solution may be injected into a body cavity, such as a thoracic cavity, for example, to arrest a noncompressible hemorrhage (e.g., tension pneumothorax). In another embodiment, administration of the solution may prevent, for example, hypovolemic shock and/or hypovolemic cardiac arrest, two of the more common morbidities associated with uncontrolled hemorrhage. In some embodiments, the solution may be administered in a pre-hospital setting (e.g., at the site of an automobile accident) and/or in austere environments (e.g., on a battlefield).

Some aspects of the disclosure relate to a thermoresponsive polymer. In some cases, a thermoresponsive polymer dissolved in a solution may reversibly transition into a gel (e.g., a hydrogel) when heated to above a certain temperature. In some cases, the thermoresponsive polymer may be a linear polymer. In certain cases, the polymer may be a branched polymer (i.e., a main chain with one or more substituent side chains or branches). Non-limiting examples of branched polymers include star polymers, comb polymers, polymer brushes, dendronized polymers, ladder polymers, and dendrimers. In some embodiments, the thermoresponsive polymer comprises a copolymer. Exemplary embodiments include alternating copolymers, random copolymers, gradient copolymers, block copolymers, graft copolymers, etc.

In some cases, a thermoresponsive polymer comprises a homopolymer comprising a poly(acrylamide), such as, for example, poly(N-isopropyl acrylamide), poly(N,N-diethyl acrylamide), poly(N-ethylmethacrylamide), poly(N-iso-propylmethacrylamide), poly(N-cyclopropylacrylamide), poly-N-(2,2-dimethyl-1,2-dioxan-5yl) methacrylamide, poly-N-(2,2-dimethyl-1,3-dioxan-5-yl) acrylamide. In other embodiments, the thermoresponsive polymer comprises a poly(caprolactam), such as, for example, poly(N-vinylcaprolactam). The thermoresponsive polymer, in some embodiments, may comprise a poly(vinyl ether), such as, for example, poly(methyl vinyl ether) and/or (2-ethoxyethyl vinyl ether). Other homopolymers are also possible (e.g., an elastin side chain polymer)

In certain cases, a thermoresponsive polymer comprises a copolymer. Exemplary embodiments include alternating copolymers, random copolymers, gradient copolymers, block copolymers, graft copolymers, etc. In some embodiments, the copolymer comprises two or more monomers. In some cases, the monomer comprises an acrylamide, a N-vinyl caprolactam, a vinyl ethers, or any combination thereof.

In some cases, a thermoresponsive polymer comprises a branched polymer (i.e., a main chain with one or more substituent side chains or branches). Non-limiting examples of branched polymers include star polymers, comb polymers, polymer brushes, dendronized polymers, ladder polymers, and dendrimers. In some embodiments, the branched polymer may be a homopolymer or a copolymer, etc. In some embodiments, the branched polymer comprises one or more monomers. Examples of monomer include acrylamide, a N-vinyl caprolactam, a vinyl ethers, or the like.

In some cases, a thermoresponsive polymer may be dissolved in a solution, wherein the solution may exhibit a lower critical solution temperature (LCST), which is the critical temperature below which the components of a mixture are miscible for all compositions (i.e., above this temperature, the solution may phase separate, for example, into a liquid polymer poor phase and a vitrified polymer-rich phase). Macroscopically, the solution may appear to transition from a liquid into a solid (e.g., a hydrogel). In some embodiments, the solution may comprise an LCST of greater than or equal to 25° C., greater than or equal to 27° C. greater than or equal to 30° C., greater than or equal to 32° C., greater than or equal to 35° C., greater than or equal to 37° C., etc. In other embodiments, the solution may comprise an LCST of less than or equal to 37° C., less than or equal to 35° C., less than or equal to 32° C., less than or equal to 30° C., less than or equal to 27° C., less than or equal to 25° C., etc. Combinations of these temperature ranges are also possible; for example, the LCST may be between 25° C. and 37° C., or between 32° C. and 35° C., etc.

In certain cases, a solution comprising a thermoresponsive polymer may have an upper critical solution temperature (UCST), which is the critical temperature above which the components of a mixture are miscible in all proportions. In some embodiments, the solution may comprise a UCST of greater than or equal to 37° C., greater than or equal to 40° C., greater than or equal to 45° C., greater than or equal to 50° C., greater than or equal to 55° C., greater than or equal to 60° C., etc. In other embodiments, the solution may comprise an UCST of less than or equal to 60° C., less than or equal to 55° C., less than or equal to 50° C. less than or equal to 45° C., less than or equal to 40° C., less than or equal to 37° C., etc. Combinations of these temperature ranges are also possible; for example, the UCST may be between 37° C. and 40° C., or between 40° C. and 50° C., etc.

In some embodiments, a thermoresponsive polymer may be dissolved in an aqueous solution. The solution may optionally comprise a plurality of nanoparticles. In some embodiments, the polymer may be dissolved in the solution at any concentration up to the solubility limit of the polymer in the aqueous solution. In some cases, the polymer is dissolved in the solution at a concentration of between 0.1% (wt/wt) to about 10% (wt/wt). In some embodiments, the polymer is dissolved in the solution at a concentration greater than or equal to 0.1% (wt/wt), greater than or equal to 0.5% (wt/wt), greater than or equal to 1% (wt/wt), greater than or equal to 2.5% (wt/wt), greater than or equal to 5% (wt/wt), greater than or equal to 7.5% (wt/wt), greater than or equal to 10% (wt/wt). In other embodiments, the polymer is dissolved in the solution at a concentration less than or equal to 10% (wt/wt), less than or equal to 7.5% (wt/wt), less than or equal to 5% (wt/wt), less than or equal to 2.5% (wt/wt), less than or equal to 1% (wt/wt), less than or equal to 0.5% (wt/wt), less than or equal to 0.1% (wt/wt), etc. Combinations of these percentages are also possible in certain embodiments.

In some embodiments, a thermoresponsive polymer may have a number average molecular weight (i.e., mole fraction of molecules in a polymer sample) of between 1000 and 400,000. In some embodiments, the number average molecular weight is greater than 1000, greater than 5000, greater than 10,000, greater than 50,000, greater than 100,000, greater than 200,000, greater than 300,000, greater than 400,000, etc. In other embodiments, the number average molecular weight is less than or equal to 400,000, less than or equal to 300,000, less than or equal to 200,000, less than or equal to 100,000, less than or equal to 50,000, less than or equal to 10,000, less than or equal to 5000, less than or equal to 1000, etc. Combinations of these are possible in certain embodiments.

In certain embodiments, the thermoresponsive polymer may have a weight average molecular weight (i.e., the weight fraction of molecules in a polymer sample) of between 1000 and 400,000. In some embodiments, the weight average molecular weight is greater than 1000, greater than 5000, greater than 10,000, greater than 50,000, greater than 100,000, greater than 200,000, greater than 300,000, greater than 400,000, etc. In other embodiments, the weight average molecular weight is less than or equal to 400,000, less than or equal to 300,000, less than or equal to 200,000, less than or equal to 100,000, less than or equal to 50,000, less than or equal to 10,000, less than or equal to 5000, less than or equal to 1000, etc. Combinations of these are possible in certain embodiments.

In some embodiments, a thermoresponsive polymer may have a polydispersity index (i.e., the ratio of the weight average molecular weight to the number average molecular weight) of between 1 and 5. In some cases, the polydispersity index is greater than or equal to 1, greater than or equal to 1.1, greater than or equal to 1.2, greater than or equal to 1.3, greater than or equal to 1.4, greater than or equal to 1.5, greater than or equal to 1.6, greater than or equal to 1.7, greater than or equal to 1.8, greater than or equal to 1.9, greater than or equal to 2, greater than or equal to 3, greater than or equal to 4, greater than or equal to 5, etc. In other cases, the polydispersity index is less than or equal to 5, less than or equal to 4, less than or equal to 3, less than or equal to 2, less than or equal to 1.9, less than or equal to 1.8, less than or equal to 1.7, less than or equal to 1.6, less than or equal to 1.5, less than or equal to 1.4, less than or equal to 1.3, less than or equal to 1.2, less than or equal to 1.1, less than or equal to 1.0, etc. Combinations of these are possible in certain embodiments.

In some embodiments, a thermoresponsive polymer (e.g., homopolymer, branched polymer, and/or block polymer) may be purchased through a commercial vendor (e.g., Sigma, BASF, etc.) or synthesized using any method known to those of skill in the art. For example, in some embodiments, a step-growth polymerization reaction may be used to produce the thermo-responsive polymer; in other cases, a chain-growth polymerization reaction (e.g., free radical polymerization, ionic polymerization, coordination polymerization, living polymerization, ring-opening polymerization, and reversible-deactivation polymerization) may be used to produce the thermo-responsive polymer. Other synthetic routes are also possible, e.g., polycondensation and addition polymerization.

Some aspects of the disclosure relate to a plurality of charged nanoparticles. In some cases, a nanoparticle may have a maximum dimension of between 1 nm to 1000 nm. A nanoparticle may be spherical or nonspherical. In some embodiments, the maximum dimension of the nanoparticle is greater than or equal to 1 nm, greater than or equal to 10 nm, greater than or equal to 50 nm, greater than or equal to 100 nm, greater than or equal to 500 nm, greater than or equal to 750 nm, greater than or equal to 1000 nm, etc. In other embodiments, the dimension of the nanoparticle is less than or equal to 1000 nm, less than or equal to 750 nm, less than or equal to 500 nm, less than or equal to 100 nm, less than or equal to 50 nm, less than or equal to 10 nm, less than or equal to 1 nm, etc.

In some cases, a plurality of charged nanoparticles may have a variety of aspect ratios. For example, in some embodiments, the plurality of charged nanoparticles may have an aspect ratio of greater than or equal to 1:0.1, greater than or equal to 1:0.5, greater than or equal to 1:0.75, greater than or equal to 1:1, greater than or equal to 0.75:1, greater than or equal to 0.5:1, greater than or equal to 0.1:1, etc. In other embodiments, the plurality of charged nanoparticles may have an aspect ratio of less than or equal to 0.1:1, less than or equal to 0.5:1, less than or equal to 0.75:1, less than or equal to 1:1, less than or equal to 1:0.75, less than or equal to 1:0.5, less than or equal to 1:0.1, etc.

In some embodiments, a plurality of charged nanoparticles may exist, for example, as a nanocluster (i.e., agglomerate of nanoparticles), nanopowders (i.e., agglomerates of ultrafine particles), single crystals, single-domain ultrafine particles (e.g., nanocrystals), and/or colloids (e.g., solid nanoparticles dispersed or suspended an aqueous phase).

In some cases, a plurality of charged nanoparticles may have a variety of shapes. For example, in some embodiments, the shape of the plurality of charged nanoparticles may comprise a nanosphere, a nanorod, a nanochain, a nanostar, a nanoflower, nanoreef, nanowhisker, nanofiber, nanoshell, nanocage, and nanobox. In other cases, the shape of the plurality of charged nanoparticles may comprises a sphere, disc, cylinder, rod, cube, triangle, octahedron, hexagon, pentagon, flower, platelet, cluster, etc. Other shapes and/or combinations of shapes are also possible.

Examples of charged nanoparticles that may be present include synthetic smectic clay nanoparticles (e.g., Laponite, lithium sodium magnesium silicate particles, etc.), ceramic nanoparticles (e.g., oxides, carbides, carbonates, and phosphates), metal nanoparticles (e.g., iron oxide), polymer nanoparticles (e.g., PLGA-PEG), lipid nanoparticles (liposomes, solid lipid nanoparticles), semi-conductor nanoparticles (e.g., GaN, GaP, InP, InAs, ZnO, ZnS, CdS, CdSe, CdTe, etc.), and carbon-based nanoparticles (e.g., carbon nanotubes and fullerenes), etc. One or more than one type of charged nanoparticle may be present, including these and/or other types of charged nanoparticles.

In some embodiments, the surface area to volume ratio of a plurality of charged nanoparticles may vary. For example, in some cases, the surface area to volume ratio of the plurality of charged nanoparticles may be between 103:1 to about 108:1. In some embodiments, the surface area to volume ratio of the plurality of charged nanoparticles is greater than or equal to 103:1, greater than or equal to 104:1, greater than or equal to 105:1, greater than or equal to 106:1, greater than or equal to 107:1, greater than or equal to 108:1, etc. In other cases, the surface area to volume ratio of the plurality of charged nanoparticles is less than or equal to 108:1, less than or equal to 107:1, less than or equal to 106:1, less than or equal to 105:1, less than or equal to 104:1, less than or equal to 103:1, etc. Combinations of any of these ranges are also possible.

In some embodiments, a plurality of charged nanoparticles may be dispersed in an aqueous solution. In some cases, the nanoparticles are dispersed in the solution at a concentration of between 0.1% (wt/wt) to about 10% (wt/wt). In some embodiments, the nanoparticles are dispersed in the solution at a concentration greater than or equal to 0.1% (wt/wt), greater than or equal to 0.5% (wt/wt), greater than or equal to 1% (wt/wt), greater than or equal to 2.5% (wt/wt), greater than or equal to 5% (wt/wt), greater than or equal to 7.5% (wt/wt), greater than or equal to 10% (wt/wt). In other embodiments, the nanoparticles are dispersed in the solution at a concentration less than or equal to 10% (wt/wt), less than or equal to 7.5% (wt/wt), less than or equal to 5% (wt/wt), less than or equal to 2.5% (wt/wt), less than or equal to 1% (wt/wt), less than or equal to 0.5% (wt/wt), less than or equal to 0.1% (wt/wt), etc. Combinations of any of these ranges are also possible.

In some instances, a plurality of charged nanoparticles may be dispersed in a solution comprising one or more salts. In some embodiments, solutions with salt concentrations above a critical coagulation concentration (CCC) may destabilize the nanoparticle colloidal dispersion, causing agglomeration of the nanoparticles. The CCC occurs at the inflection point of a plot of the colloidal stability ratio versus log salt concentration. In some embodiments, the CCC may be greater than or equal to 0.01×10⁻³ mol/L, greater than or equal to 0.5×10⁻³ mol/L, greater than or equal to 2×10⁻³ mol/L, greater than or equal to 10×10⁻³ mol/L, greater than or equal to 20×10⁻³ mol/L, greater than or equal to 50×10⁻³ mol/L, greater than or equal to 100×10⁻³ mol/L, greater than or equal to 200×10⁻³ mol/L, greater than or equal to 300×10⁻³ mol/L. In other embodiments, the CCC may be less than or equal to 300×10⁻³ mol/L, less than or equal to 200×10⁻³ mol/L, less than or equal to 100×10⁻³ mol/L, less than or equal to 50×10⁻³ mol/L, less than or equal to 20×10⁻³ mol/L, less than or equal to 10×10⁻³ mol/L, less than or equal to 2×10⁻³ mol/L, less than or equal to 0.5×10⁻³ mol/L, less than or equal to 0.01×10⁻³ mol/L, etc. Combinations of any of these ranges are also possible.

In some embodiments, a plurality of charged nanoparticles (e.g., polymer, metal, semiconductor, etc.), may be purchased through a commercial vendor, or synthesized using any suitable method. Artificial nanoparticles may be created from virtually any solid or liquid material, including metals, dielectrics, and semiconductors, etc. In some cases, the nanoparticles may be internally homogeneous (solid nanoparticle) or heterogenous (e.g., core-shell structure). Exemplary methods for synthesizing nanoparticles include gas condensation reactions (e.g., plasma condensation or inert gas condensation), attrition (e.g., grinding solid particles in a mill), chemical precipitation, ion implantation, pyrolysis, hydrothermal synthesis, and biosynthesis.

In certain embodiments, a plurality of charged nanoparticles may comprise a coating, for example, to enhance stability in a colloidal dispersion. In some embodiments, the coating may comprise grafting one or more hydrophilic polymers (e.g., polyethylene glycol), zwitterionic polymers (e.g., trimethylamine-n-oxide), and/or hydrophobic polymers (e.g., perfluorocarbons) to one or more surfaces of the layer (e.g., polymer and/or metallic surfaces). In some embodiments, grafting perfluorocarbon like moieties to the surface may render the layer hydrophobic or superhydrophobic. As another example, grafting PEG-like and TMAO-like moieties to the surface may render the nanoparticle hydrophilic or superhydrophilic. In some embodiments, the coating may comprise increased surface charge densities, for example, caused by oxygen plasma etching and/or layer by layer deposition of oppositely charged polymers (e.g., poly-L-lysine and polyacrylic acid).

In some embodiments, a coating may comprise one or more therapeutic compounds. In some embodiments, the therapeutic compound may comprise an antimicrobial compound, for example, to prevent infection within a site of hemorrhage. In some embodiments, the antimicrobial compound comprises a penicillin. Non-limiting examples include penicillin V, penicillin G, amoxicillin, amoxicillin/clavulonate, ampicillin, nafcillin, oxacillin, dicloxacillin, piperacillin, pipercillin/tazobactam, and the like. In some embodiments, the antimicrobial compound comprises a macrolide. Examples include, but are not limited to, azithromycin, clarithromycin, fidaxomicin, erythromycin, telithromycin, and the like. In some embodiments, the antimicrobial compound comprises a cephalosporin. Examples include, but are not limited to, cefacetril, cefradin, cefroxadin, cefaloglycin, cefaclor, cefalexin, cefadroxil, cefatrizin, cefazedon, cefapirin, ceftezol, cefazolin, cefazaflur, cefalotin, cefaloridin, cefalonium, and the like. In some embodiments, the antimicrobial compound comprises a fluoroquinolone. Examples include balofloxacin, grepafloxacin, levofloxacin, pazufloxacin, sparfloxacin, temafloxacin, clinafloxacin, gatifloxacin, moxifloxacin, sitafloxacin, prulifloxacin, besifloxacin, delafloxacin, and the like. In some embodiments, the antimicrobial compound comprises a beta-lactam. Examples include penams, carbapenams, clavams, penems, carbapenems, cephems, carbacephems, oxacephems, monobactams, and the like. Combinations are also possible (e.g., the coating may comprise a penicillin and a beta-lactam or a fluoroquinolone and a cephalosporin, etc.).

Some aspects of the disclosure relate to injecting a solution comprising a thermoresponsive polymer and a plurality of charged particles into a site of hemorrhage. In certain cases, injecting the solution causes a plug to form within the site of hemorrhage. In some embodiments, injecting the solution causes the thermoresponsive polymer to form a hydrogel comprising the thermoresponsive polymer. The hydrogel encapsulates the charged nanoparticles in certain embodiments. In some cases, the hydrogel may be porous, which may allow for transport of various blood components (e.g., platelets and clotting factors) through the hydrogel. Blood components (e.g., platelets, clotting factors, etc.,) may, in some instances, come into contact with the charged nanoparticles embedded within the porous hydrogel, causing a blood clot to form within and around the pores, such that the blood clot is intertwined with the hydrogel in some embodiments to form a plug.

In some embodiments, injecting a solution comprising a thermoresponsive polymer and a plurality of charged nanoparticles into a site of hemorrhage accelerates the rate at which a blood clot forms. In some cases, injecting the solution may also improve the viscoelastic properties of the blood clot (e.g., plug), such that the blood clot remains within the site of hemorrhage when exposed to physiologically relevant hydrostatic pressures (e.g., between 50 mmHg and 200 mmHg), which may slow the rate of blood loss from the site of hemorrhage.

The viscoelastic properties of a blood clot may be determined using, for example, thromboclastography (e.g., TEG), ROTEM, rotational thromboelastometry) or other techniques known to those of ordinary skill in the art for determining blood clot properties, such as rheology. For instance, TEG and/or ROTEM are viscoelastic hemostatic assays that measure the global viscoelastic properties of whole blood clot formation under low shear stress. TEG parameters, for example, provide information such as (1) reaction time or R-value, which is the time of latency from the start of the test to the initial fibrin formation at a predefined amplitude of 2 mm; (2) the kinetics or K value, which is the time taken to achieve a certain level of clot strength (e.g., amplitude of 20 mm); (3) the alpha angle, which measures the speed at which fibrin build up and crosslinking takes place (assesses the rate of clot formation); (4) the time to maximum amplitude; (5) maximum amplitude in millimeters or MA, which represents the ultimate strength of the fibrin clot and its overall stability; or (6) the LY30, which is the percent decrease in amplitude at 30 minutes post MA (characterizes lysis or degradation of the clot).

In some embodiments, injecting a solution comprising a thermoresponsive polymer and a plurality of charged nanoparticles into a site of hemorrhage creates a plug. The plug may comprise a hydrogel comprising the thermoresponsive polymer, the charged nanoparticles, and a blood clot. In some embodiments, the plug comprises a reaction time (e.g., R value) of between 4 min and 8 min; a kinetics of formation (K value) time of between 1 min and 4 min; an alpha angle value of between 47 degrees and 74 degrees; a maximum amplitude (MA) value of between 55 mm and 73 mm; and a LY30% value of between 0% and 8%. The following examples are intended to illustrate certain embodiments of the present disclosure, but do not exemplify the full scope of the disclosure.

U.S. Provisional Patent Application Ser. No. 63/348,119, filed Jun. 2, 2022, entitled “Systems and Methods for Shear-Thinning Hemostats,” by Mecwan, et al., is incorporated herein by reference in its entirety.

### Example 1

### 1.1. Abstract

Hemorrhage and sepsis are the two leading causes of death following battlefield injuries. Although several hemostats in the form of hydrogels, sheets, sponges, and powder are commercially available, they do not meet all the requirements necessary for combat injuries; specifically hemorrhage control. As described herein, thermo-responsive shear-thinning hydrogels (T-STH), composed of poly(N-isopropyl acrylamide) and Laponite were engineered as injectable hemostatic agents with the ability to deliver a therapeutic agent. T-STHs form physiologically stable hydrogels and can be easily injected through a syringe and needle, and exhibit rapid mechanical recovery. More importantly, T-STHs decrease in vitro blood clotting times over 50% and significantly prevent blood loss in an ex vivo bleeding model at different blood flow rates (1 mL/min and 5 mL/min). Additionally, T-STHs can deliver therapeutic doses of antibiotics for 72 hours in the applied site locally to avoid pathogen invasion. Taken together, poly(N-isopropyl acrylamide) and Laponite-based T-STHs disclosed herein can be used as an injectable hemostat to treat external hemorrhages as well be used as an effective drug delivery vehicle for the delivery of antibiotics to prevent infections.

### 1.2. Introduction

Most battlefield injury-related mortalities occur in the pre-medical treatment facility environment before the injured soldier or victim can ever reach a hospital or a surgeon. Of these, hemorrhage is responsible for most trauma-related mortality and is the leading cause of death on the battlefield. Post-traumatic sepsis following hemorrhage is another cause of mortality but can be prevented through early intervention and treatment. Therefore, an ideal hemostat for battlefield injuries and pre-medical treatment facility hemorrhage control should have the following characteristics: 1) quick and adequate hemostasis in a wide range of injuries and wounds, 2) sustained hemostasis for several hours with the ability to deliver antibiotics in situations of delayed evacuation, 3) easy removal without leaving any residues in the injury or wound, 4) ready to use and easy administration by a layperson with little to no training. 5) easy to manufacture and sterilize with low costs, 6) easily stored with prolonged stability even under extreme climate conditions, and 7) good biocompatibility with no adverse effects.

Since 2008, QuikClot Combat Gauze® has been the gold standard for combat casualties by all US military branches. It is a gauze made of rayon and polyester blend loaded with kaolin, which helps control and stop bleeding by activating factor XII (i.e., it stimulates clot formation). This product is physically packed into a wound bed to gain control over the site of hemorrhage; however, it has a limited capacity to absorb fluid and is inefficient at forming a solid robust clot capable of withstanding the hydrostatic pressures and forces exerted by a lacerated vessel (e.g., femoral artery). Further, this device cannot be used to apply pressure to noncompressible hemorrhages, e.g., tension pneumothorax, without opening the thoracic cavity, thus limiting their use to external wound management.

A variety of other hemostats in the form of hydrogels, sheets, sponges, and powders have also been reported in the literature, but none of them meet the requirements for clinical translation (as defined above). Therefore, there is a need for innovative multifunctional hemostats that can be used to manage blood loss at a site of hemorrhage via simple injection.

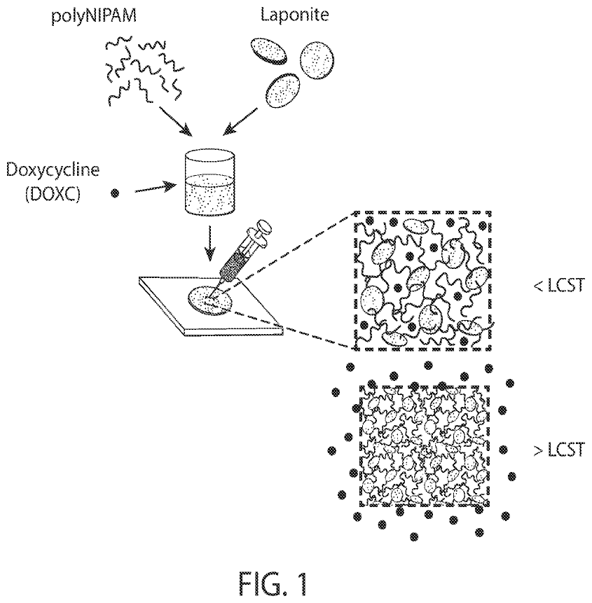

As presented herein, a short-term hemostat was developed to treat hemorrhages and infections associated with traumatic injuries on the battlefield and in emergency response situations. To this end, an injectable sheer-thinning thermoresponsive hydrogel (T-STH) made up of polyNIPAM and Laponite was engineered to function as a hemostat plug at physiological conditions (FIG. 1). Here, the effect of varying polyNIPAM concentrations on the rheological properties and injectability of the T-STHs was assessed. Then, the thermoresponsive nature of the T-STHs and their ability to function as a hemostat and significance in blood coagulation was evaluated via an in vitro and an ex vivo bleeding model. Subsequently, the thermoresponsive T-STH was loaded with doxycycline (DOXC), a broad-spectrum antibiotic, and their antibacterial activity against both S. aureus (gram-positive) and E. coli (gram-negative) was tested.

### 1.3 Materials and Methods

### 1.3.1. Materials

For the preparation of T-STHs, polyNIPAM (MW 40,000 Da) was purchased from Sigma Aldrich (Cat #535311), and silicate nanoplatelets or Laponite® XLG was purchased from BYK Additives Ltd. Citrated human whole blood was purchased from ZenBio (SER-WB). The blood type was O+ for the hemocompatibility studies and belonged to a 24-year-old African American male with a 35.7 BMI. For the ex vivo studies, the blood type was also O+ and belonged to a 53-year-old Hispanic male with a 25.68 BMI. In addition, calcium chloride (CaCl₂)) solution (0.1 M) and 0.9% saline solution was purchased from Spectrum Chemical Manufacturing Corp (C-092) and Teknova (S5815), respectively. Finally, for the antibiotic loading and delivery studies, doxycycline hyclate (≥98%, HPLC grade) was purchased from Sigma Aldrich (D9891).

### 1.3.2. Thermoresponsive STH (T-STH) Formulations and SEM Imaging

Stock solutions of polyNIPAM (20% w/v) and Laponite (12% w/v) were prepared in Milli-Q water (4° C.). Four different T-STH formulations were prepared by vortexing appropriate ratios of the PolyNIPAM stock, Laponite stock, and Milli-Q water. The T-STH formulations had a general formula xNyL, where x=2.5, 5, 7.5 and 10% w/w of polyNIPAM, and y=3% w/w of Laponite. The vortexing was done 3 times, at 3000 rpm for 5 min using a SpeedMixer™ (DAC 150.1 FVZ), and the T-STHs were stored overnight at 4° C. before use.

The temperature-dependent phase transition of the T-STH was observed by taking digital images of the samples at room temperature and at 37° C. The morphology of dried T-STH with various compositions was characterized by scanning electron microscopy (SEM) (FEI Quanta 200, Hillsboro, OR). All samples for SEM imaging were kept at −80° C. for 24 h, freeze-dried for 48 h, and mounted onto a metallic stub using double-sided conductive carbon tape. Samples were then sputter-coated in an Ar atmosphere with an Au—Pd target at a peak current of 15 uA for 5 min and subsequently imaged using an accelerating voltage of 15-25 kV.

### 1.3.3. Rheological Analysis

T-STH shear rate, frequency sweeps, and recoverability were analyzed according to previously reported protocols with minor modifications. An Anton Paar MCR 302 rheometer was used for mechanical testing, and the data was recorded via Anton Paar Rheocompass software. Shear stress, viscosity, and storage moduli were measured with a 25 mm diameter parallel plate geometry with a rough surface, and a gap height of 500 μm was used. Mineral oil was added around the plate to prevent water evaporation from the T-STH once the sample was loaded. All T-STHs were equilibrated for 5 mins before testing, followed by a 2 min steady shear at 10 s⁻¹. Steady shear rate sweeps investigated the shear-thinning properties of the samples at 25 and 37° C., and the viscosities of the materials were measured as a function of shear rate. Step-rate time-sweep was performed to investigate the thixotropic recovery properties of the samples between low shear strain (1%) and high shear strain (100%) at 25 and 37° C. Oscillation amplitude sweep and frequency sweeps were applied to measure the storage modulus (G′) and loss modulus (G″) of the samples at 37° C. Temperature sweeps from 15 to 45° C. at a heating rate of 1° C./min were carried out to measure gelation temperature, whereas time sweeps at 37° C. were performed to investigate the gelation kinetics.

### 1.3.4 Injectability of T-STH

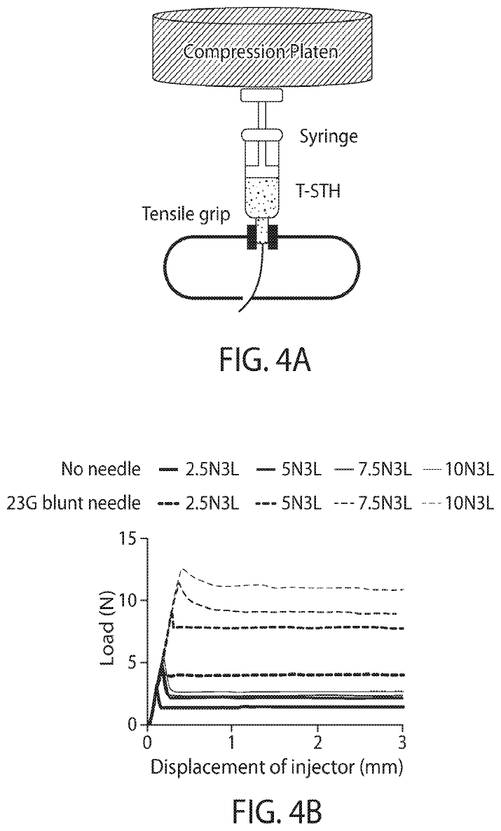

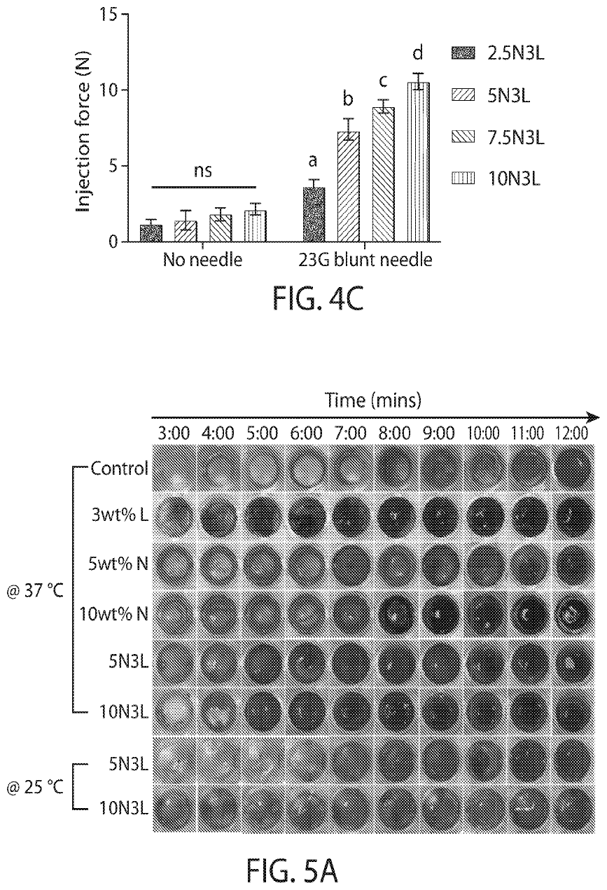

The injectability of T-STHs was analyzed using an Instron (Model 5542). Briefly, the T-STH was added to a 3 mL syringe and centrifuged at 1000 rpm for 5 mins to pack the T-STH within each syringe. The T-STHs were then injected through the syringe either with no needle or a 23G blunt needle (BD biosciences) using standard Luer-lock fittings. The syringe plunger was depressed using an upper compressive platen. The housing of the syringe or needle was fitted into a lower tensile grip to prevent movement during the experiment (FIG. 4A). An injection rate of 33.33 mL/min was used for these tests, and the force on the plunger was measured with a 100 N load cell and recorded using Bluchill version 3 software. The plateau's average injection force (N) was obtained by quintuple measurements of three identical compositions of each T-STH formulation.

### 1.3.5 Degradation of T-STH

T-STH degradation was performed either in PBS or human plasma. First, human plasma was separated from citrated human whole blood by centrifugation at 3000 rpm for 15 mins (Beckman Coulter Allegra™ 6R centrifuge) and stored at −80° C. until use. Next, 0.2 mL of each T-STH formulation was injected into 1.5 mL Eppendorf tubes, centrifuged to flatten, and weighed (˜175-200 mg). Then, 1 mL of pre-warmed PBS or human plasma was added to each sample and placed in a benchtop orbital shaker at 37° C. with constant shaking at 100 rpm (Barnstead Lab-Line MaxQ 4000). After incubation for 1, 3, 6, 10, 24, 30, and 48 h, the PBS or human plasma was removed, and the remaining T-STH was weighed. Each sample was replaced with either fresh PBS or human plasma and returned to the incubator. The relative weight percentage of each T-STH was defined as W %=(Wr/W0)×100, where Wr is the weight of the remaining T-STH at various time points, and W0 is the weight of T-STH at the initial state. T-STH degradation studies in both PBS and human plasma were performed in triplicates, and mass remaining (%) is reported as mean±standard deviation of the replicates.

### 1.3.6 Cytotoxicity Studies

NIH/3T3 cells (ATCC, CRL 1658) were cultured in Dulbecco's Modified Eagle Medium (Gibco, 1165092) and supplemented with 10% fetal bovine serum, 50 μg/mL streptomycin, and 50 U/mL penicillin in 5% CO2 at 37° C. Cells were seeded in a 24-well plate (1×10⁴ cells/well) and grown for 24 h with 1 mL of complete growth media. Next, 0.2 mL of T-STH was injected into transwell inserts (Costar, 3396) and sterilized via UV sterilization for 30 mins. The eluates from the T-STH samples in the transwell inserts were then transferred to the 24-well plate with NIH 3T3 cells, and fresh complete growth media was added (1 mL) to the T-STH samples in the transwell inserts and changed daily throughout the experiment. Cytotoxicity was assessed at days 1, 3, and 5 using PrestoBlue™ Cell Viability Reagent (A13261, ThermoFisher) following the manufacturer's protocol. Transwell inserts without any T-STH were used as controls. All data were normalized to the controls and reported as mean±standard error means of all replicates.

### 1.3.7 Clotting Time

Clotting times were measured according to previously reported protocols with minor variations. A volume of 630 μL of citrated whole blood was pipetted into a 1.5 mL Eppendorf tube, and a volume of 70 μL of 0.1 M CaCl2 was then added, followed by vortexing for 10 s, to reactivate the blood. Immediately, 0.2 mL of the blood was transferred to 24-well plates. At every minute between 3 and 12 mins, each well was washed with 0.9% saline solution to halt clotting. The liquid was immediately aspirated, and the samples were washed repeatedly until the wash solution was clear, indicating complete removal of blood components. For the T-STH samples, 0.2 mL of T-STH was injected into wells of a 24-well plate and centrifuged at 2000 rpm for 10 mins to evenly flatten each sample. The final clotting time was determined when a uniform clot was formed, with no change in clot size in subsequent wells. These clotting time studies were performed at room temperature (˜25° C.) and 37° C. in triplicates for each time point.

### 1.3.8 Thrombus Weights

Thrombus weights were determined according to previously published protocols with minor variations. Briefly, 0.1 mL of either 5N3L or 10N3L were injected into Eppendorf tubes, followed by a short centrifuge cycle to flatten out each sample, and the tubes containing the T-STHs were weighed. Next, citrated human whole blood was reactivated with 0.1 M CaCl2 at a 9:1 ratio and vortexed for 10 s. Immediately, 0.1 mL of the blood was pipetted into the tubes containing the T-STH, and the tubes were transferred to an Eppendorf ThermoMixer® C maintained at 37° C. under constant agitation at 300 rpm. At each time point, clotting was stopped by adding 0.2 mL of 0.109 M sodium citrate solution, followed by saline washes, until the wash solution was clear. Finally, the Eppendorf tubes with the newly formed clots were reweighed to determine the thrombus weights produced in the tubes. These studies were performed in triplicates for each time point, and the weight of the thrombus (mg) is reported as the mean±standard deviation of all replicates.

### 1.3.9 Hemolysis Ratio

Hemolysis testing was performed according to previously published protocols. Citrated human whole blood was diluted 50× with 0.9% (w/v) saline solution. First, 0.25 mL of T-STH were injected into wells of a 24-well plate and centrifuged at 2000 rpm for 10 mins to evenly flatten each sample. Next, an equal volume (0.25 mL) of diluted blood was added to each well and incubated at 37° C. under agitation (100 rpm). After 2 h, the well plate was centrifuged at 2000 rpm for 10 mins, the supernatants were transferred into wells of a 96-well plate, and the absorbance of the supernatants was read at 542 nm. Saline and Milli-Q water were used as negative and positive controls, respectively. Percent hemolysis was defined as H %=[(Aₛₐₘₚₗₑ−A_{neg})/Aₚₒₛ]×100, where Aₛₐₘₚₗₑ is the absorbance at 542 nm of the T-STH-containing supernatant, A_{neg} is the absorbance of the saline-diluted blood, and Aₚₒₛ is the absorbance of the DI water-diluted blood. These hemolysis studies were repeated twice, once in quadruplets and the second time in triplicates, and the hemolysis ratio (%) is reported as mean±standard deviation of all replicates.

### 1.3.10 Ex Vivo Bleeding Model

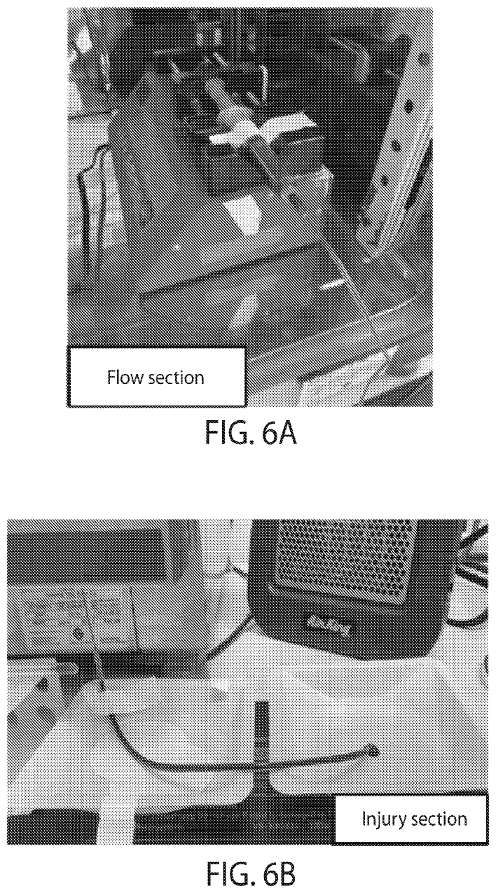

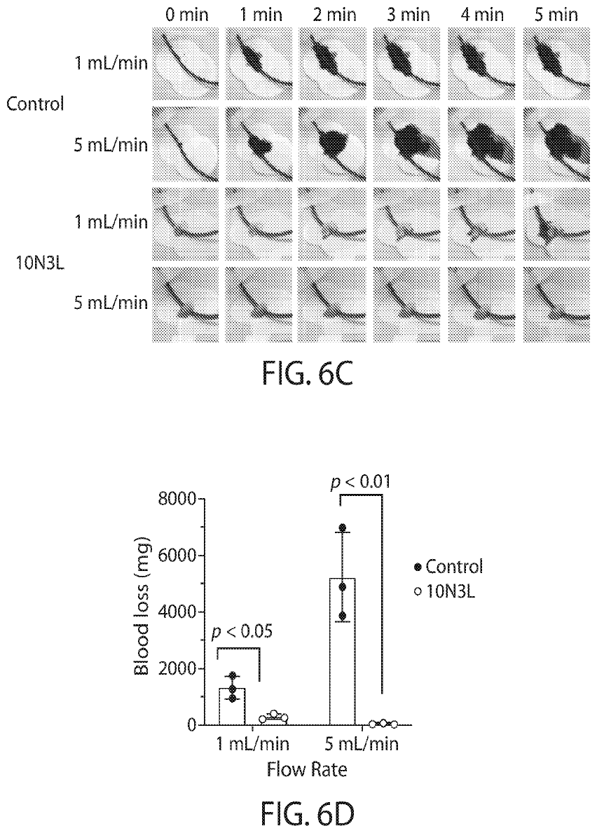

The ex vivo bleeding model setup had two sections. The “flow” section comprised a syringe pump (Braintree Scientific, Model BS8000) housed in an incubator to maintain the blood temperature at 37° C. Medical grade tubing was used as artificial blood vessels (Tygon® tubing ND-100-65; inner diameter 3/32 inches and outer diameter 5/32 inches) to flow blood from the “flow” section to the “injury” section, which was outside the incubator but placed on a heating pad (VIVOSUN reptile heat mat and digital thermostat combo) set to 37° C. and a portable heater (AIR KING non-oscillating portable electric heater) was used to maintain the ambient temperature at 37° C. An image of the ex vivo bleeding model setup can be found in FIG. 6A.

Briefly, 9 mL of citrated human whole blood was reactivated with 1 mL of 0.1 M CaCl₂ solution and vortexed for 10 s. The activated blood was gently transferred to a 10 mL syringe and was secured in the syringe pump. A 23 G needle with Tygon® tubing was then attached to the syringe. To mimic various blood flow rates in the body, these experiments were performed at two different flow rates: 1 mL/min and 5 mL/min. Once the blood started flowing at the end of the tube, an “injury” was created approximately 10 inches from the tubing end using a 1.5 mm biopsy punch to puncture the tube. The “injury” was either left untreated (control) or 1 mL of 10N3L was applied to the injury site. At every minute, from t=0 min to 5 min, a digital image was taken, and the amount of blood loss (mg) was measured by weighing the Whatman filter paper at the end of the experiment (5 mins). The ex vivo studies were performed in triplicates, and the amount of blood loss (mg) is reported as the mean±standard deviation of all replicates.

### 1.3.11 Doxycycline (DOXC) Release Study and Modeling

For the drug delivery studies, DOXC was used as a model drug. DOXC loaded T-STHs were prepared by adding DOXC to a final concentration of 1 mg/g of T-STH. Briefly, a 5 mg/mL DOXC stock solution was prepared in Milli-Q water. To prepare 5 g of DOXC loaded T-STHs, 1 mL of the DOXC stock solution was added to the polyNIPAM and silicate nanoplatelet stocks. Approximately 1 g of DOXC loaded T-STH was placed in amber vials and filled with 10 mL PBS to replicate infinite sink conditions. The vials were then placed in a 37° C. water bath with constant agitation at 100 rpm. At specific time points (0.5, 1, 2, 3, 4, 5, 6, 10, 24, 48, 72, 96, 120, 144, 168, 216, 264, 336, 432, 504, 600, and 672 h), 0.2 mL of the eluate was collected from each sample and replaced with fresh 1×PBS. The absorbance of each eluate was read at 270 nm using a UV-vis spectrophotometer and compared to a DOXC standard curve (0 μg/mL-500 μg/mL). The drug delivery studies were performed in triplicates. For drug dissolution profile modeling from DOXC-loaded T-STHs, DDSolver, an MS excel add-ins plugin, was used.

### 1.3.12 Zone of Inhibition (ZOI) Assay

ZOI assays were performed to determine the activity of the DOXC released from the T-STHs and followed previously published protocols with minor modifications. Either S. aureus (ATCC, 23235) or E. coli (ATCC, 25922) in PBS with an OD 600˜0.1 was spread evenly over sterile agar plates using a cell spreader to form bacterial lawns overnight. A 6 mm hole was bored at the center of each plate using a sterile biopsy punch (Acuderm), and 0.1 mL of DOXC loaded T-STHs was injected into the holes. The plates were then transferred to an incubator at 37° C. After 24 h, the radius of the zones was recorded, and images were taken. These ZOI assays were performed twice in duplicates, and the ZOI for each treatment group was averaged for all replicates.

Additionally, these ZOI assays were repeated using 24-well transwell inserts to determine the activity of the DOXC released from the T-STH over time. For this, 0.1 mL of DOXC loaded 5N3L or 10N3L was injected into 24-well transwell inserts and centrifuged to flatten. Again, either S. aureus or E. coli was spread evenly over sterile agar plates. An 8 mm hole was bored at the center of each plate using a sterile biopsy punch, and the transwell insert containing DOXC loaded T-STH was placed in the hole and transferred to an incubator. After 24 h, the radii of the zones were recorded, and the transwell inserts were transferred to a new sterile agar plate with bacteria freshly spread over them. This process was repeated every 24 h over 4 days. These studies were performed in triplicates.

### 1.3.13 Graphing and Statistical Analysis

GraphPad Prism 9 software was used for graphing and plotting data for this research, as well as for performing statistical analysis. When appropriate a one-way ANOVA analysis, followed by a Tukey's post-hoc analysis, was used to determine statistical differences between treatment groups. For the cytotoxicity studies, a two-way ANOVA analysis was performed to determine statistical differences between treatment groups and between days. For the ex vivo bleeding studies, an unpaired student's t-test was used to observe any statistical difference in blood loss between the control and 10N3L treatment group. In all cases, a was set to 0.05.

### 2. Results

### 2.1. T-STHs are Thermoresponsive, Injectable, and Non-Cytotoxic

Thermo-responsive hydrogels were engineered with different combinations of polyNIPAM and Laponite stock solutions. The mixtures were then homogenized via vigorous agitation using a speed mixer to prevent clumping of the Laponite during gelation. The Laponite concentration was kept constant at 0.3 g/dL, while the polyNIPAM concentration was varied from 0.25 g/dL to 1 g/dL.

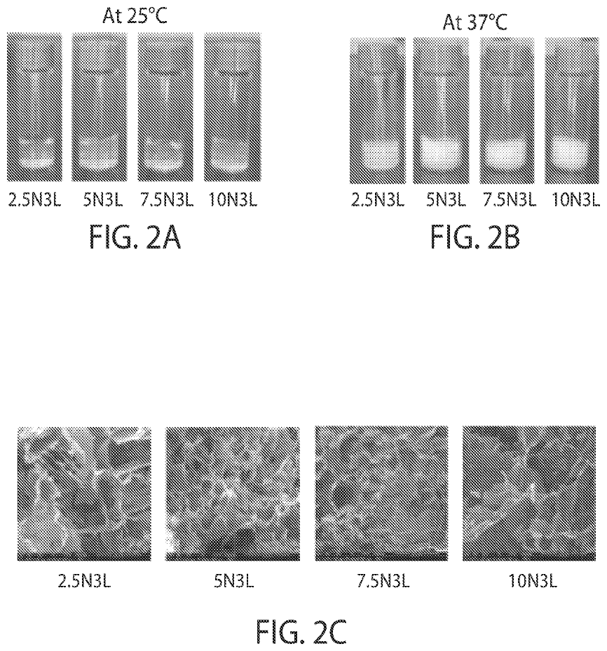

The resulting T-STHs had a clear appearance at room temperature. Increasing polyNIPAM concentration resulted in hydrogels that were more gelatinous in appearance. At 37° C., the clear appearance changes to an opaque and white hydrogel (FIG. 2A and FIG. 2B). This is a result of the phase transition of polyNIPAM from a hydrophilic state below its LCST to a hydrophobic state above its LCST. In addition, the microstructure of T-STH was observed using SEM after freeze-drying (FIG. 2C). It appeared that increasing polyNIPAM concentration within the T-STH resulted in hydrogels with lower porosity

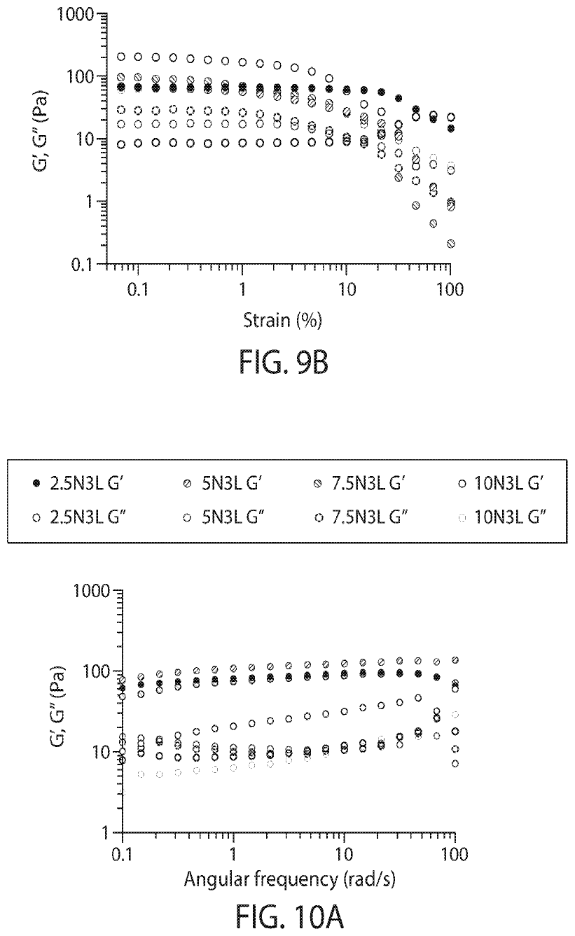

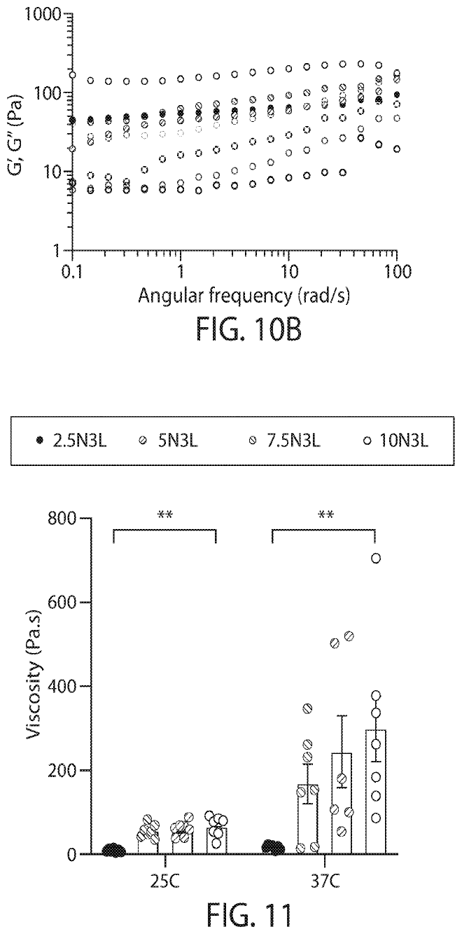

The rheological properties of the T-STHs were investigated using a rotational rheometer at both room temperature (25° C.) and body temperature (37° C.). The STH formulations showed a slightly broader linear viscoelastic range (LVER) at 25° C. as compared to 37° C., and the strain required to break the hydrogel network structure was greater than 10% at both temperatures. It should be noted that increasing the poly NIPAM concentration in the T-STHs resulted in stronger gels at 25° C. However, at 37° C., a larger polyNIPAM concentration resulted in slightly weaker gels (FIGS. 9A-9B). The frequency-dependent rheology acquired in the LVER was also investigated. (FIGS. 10A-10B). For all samples, the G′ exceeded the G″ values at both temperatures, indicating the formation of hydrogels. At 25° C., the G′ value of the T-STHs decreased with polyNIPAM concentration, whereas, at 37° C., the opposite trend was observed, indicating that polyNIPAM contributes to the mechanical strength of the T-STHs.

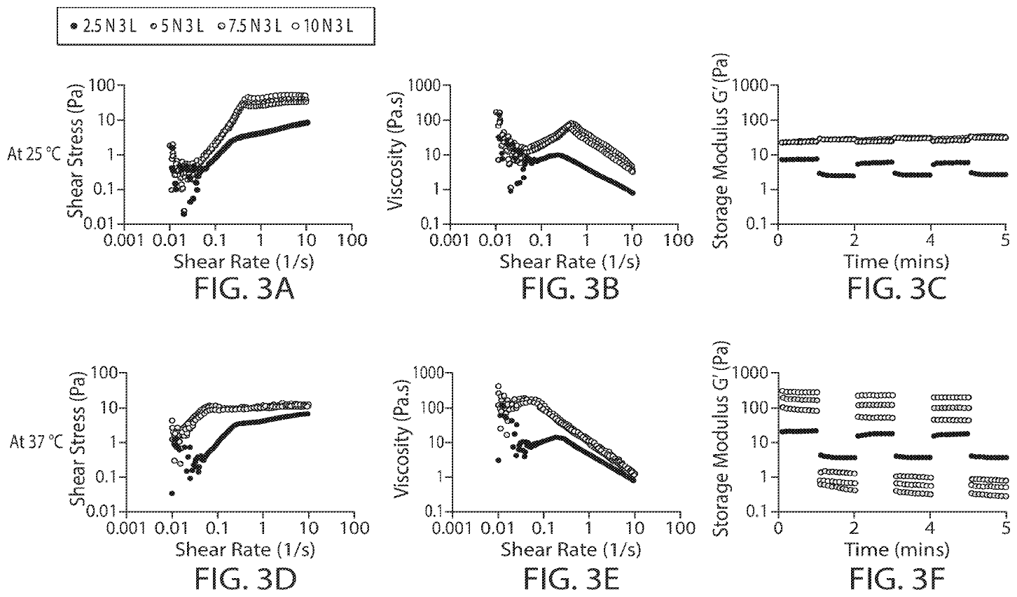

Next, the shear-thinning properties of the T-STH formulations were studied at 25° C. and 37° C. (FIGS. 3A-3F). For both temperatures, it was observed that polyNIPAM concentration did not significantly affect the viscosity of the T-STH, except in the cases of 2.5N3L and 10N3L (p<0.01). However, a change in temperature from 25° C. to 37° C. resulted in significantly more viscous STHs for each formulation (FIG. 11). Several cycles of a high strain (100% oscillatory strain) were also applied to break the network structure of the T-STH formulations, followed by low strain (1% oscillatory strain) to monitor the recovery of the storage modulus (G′) of the T-STH at both room temperature and body temperature (FIG. 3C and FIG. 3F). At 25° C., it was observed that strain had minimal effect on the G′ values of the T-STH. However, at 37° C., the effect of strain was more pronounced. It should be noted that at both temperatures, each of the T-STH formulations possessed excellent thixotropic recovery properties.

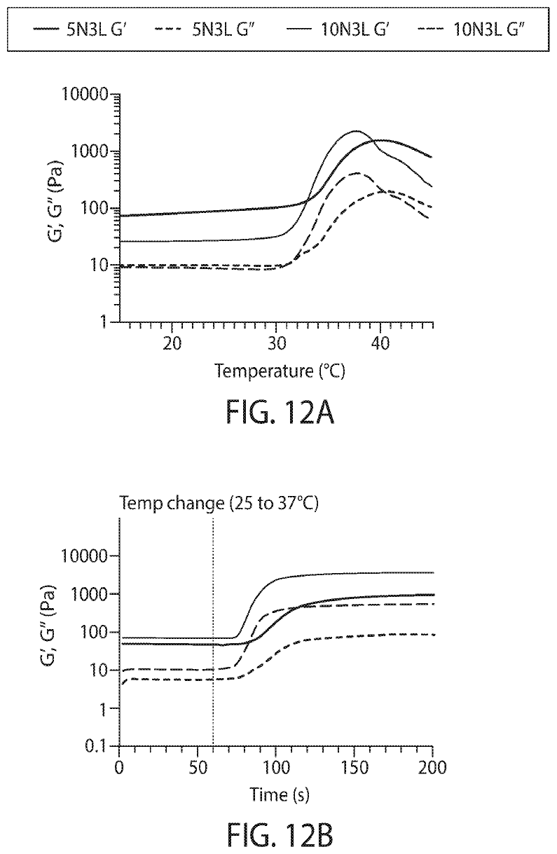

To further explore the thermoresponsive behavior of the STHs, the temperature-dependent changes in G′ and G″ values for the T-STH formulation were investigated, either through a slow temperature ramp from 15° C. to 45° C. or a sudden temperature change from 25° C. to 37° C. (FIGS. 12A-12B). The former was done to observe the transition temperature of the T-STHs, while the latter was done to recapitulate the instance in which the T-STH would be applied to a bleeding patient (37° C.) from room temperature (25° C.). The data reveals that the G′ values are always higher than the G″ values, which indicates that the T-STH is always in a hydrogel state. However, during the slow temperature ramp, it was observed that the G′ and G″ values started to increase around 31-32° C. which is comparable to the LCST of polyNIPAM (˜32° C.). Furthermore, these results indicate that in response to a sudden temperature change from 25° C. to 37° C., increasing polyNIPAM concentration results in increased G′ values as well as a faster rate change in G′ and G″ values of the T-STHs.

To assess the case of injectability of the T-STH, the injection process was replicated using a mechanical tester. The force required to inject the T-STH from 3 mL syringes either with or without a 23G blunt needle was measured (FIG. 4A). These experiments were only performed at room temperature. It was observed that in all cases, the applied force increased linearly until it plateaued at the injection force (FIG. 4B). The force required to inject the T-STH from a 3 mL syringe (without a needle) ranged between 1-2 N, with no significant differences between the T-STH formulations. However, with a 23G blunt needle, the applied force increased with increasing polyNIPAM concentration and ranged from ˜3.6 N for 2.5N3L to ˜10.6 N for 10N3L (FIG. 4C). These injection forces are easily achieved manually and do not require additional equipment for the application, making the use of these materials for the treatment of external hemorrhages in emergency medical situations efficient.

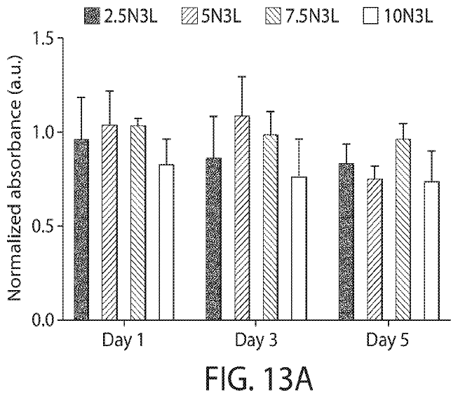

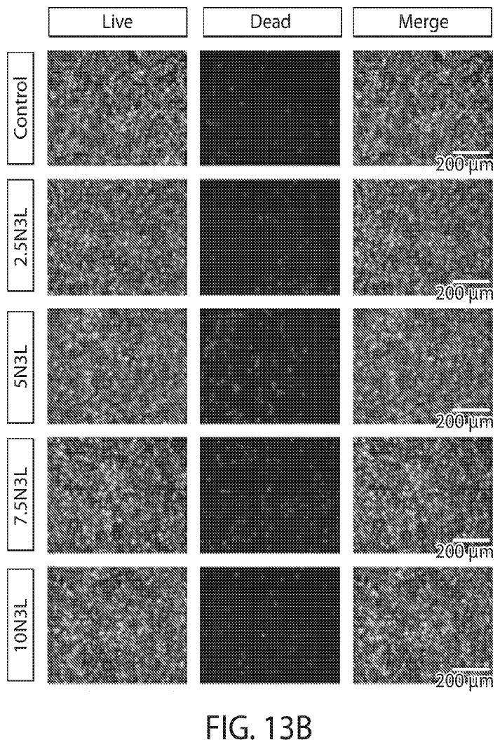

It was also demonstrated that the T-STHs were non-cytotoxic over five days as determined by both PrestoBlue™ Cell Viability and LIVE/DEAD assays (FIGS. 13A-13B). Furthermore, a 2-way ANOVA analysis of the normalized absorbance values determined no significant differences between the treatment groups or days.

### 2.2. T-STHs Promote Temperature-Dependent Coagulation In Vitro

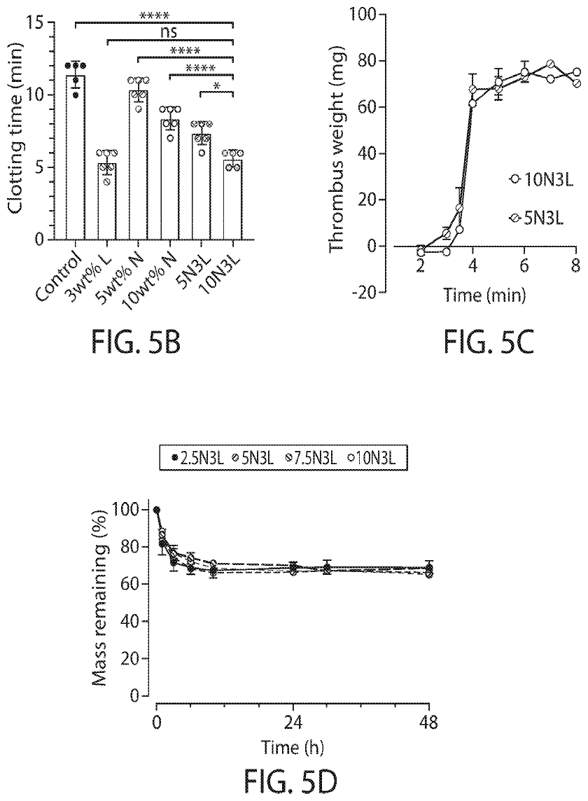

Based upon the initial characterization of the T-STHs disclosed herein, which included rheological characterization, case of injectability, and cytotoxicity testing, it was determined that 5N3L and 10N3L would be specifically focused on for blood coagulation experiments. The hemostatic ability of 5N3L and 10N3L was evaluated by monitoring the clotting time of whole blood in contact with the T-STH in 24-well plates (FIG. 5A). The blood procured for these experiments was coagulated in 11-12 mins under physiological conditions (37° C.). Under the same conditions, similar clotting times were observed for 5 wt % polyNIPAM. Increasing the polyNIPAM concentration lowered the clotting time to 8-9 mins, indicating that polyNIPAM concentration affects clotting time. It has been previously hypothesized that negatively charged Laponite interacts with platelets and other clotting factors in whole blood, which aids in coagulation. Thus, incorporating Laponite into the T-STH formulations further reduced clotting time to ˜5.6 mins for 10N3L and was comparable to 3 wt % Laponite controls, which is over 50% reduction in clotting time.

Interestingly, as seen in FIG. 5A, the clotting times for 5N3L and 10N3L at room temperature (˜23-25° C.) were comparable to whole blood. Slight color change was observed early on, which may be attributed to the Laponite; however, coagulation only occurred around 12 mins. Below the LCST of polyNIPAM (˜32° C.), it is possible the polymer is in its soluble hydrophilic state and does not allow the blood to interact with Laponite. However, above 32° C., as the polyNIPAM changes to an insoluble hydrophobic form, it entraps the blood within the polymer matrix and allows the Laponite to interact with the blood better. This elucidates that the thermoresponsive behavior of the hemostats disclosed herein aid in the coagulation of blood.

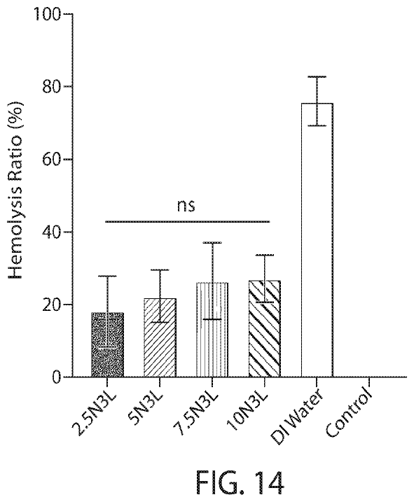

Moreover, the thrombus weights were also measured as a function of time for both 5N3L and 10N3L and observed a characteristic S-shaped curve with a clot starting to form around 3 mins and plateaued out around 6-7 mins (FIG. 5C), which correlated well with the clotting time data. Furthermore, hemolysis assays of diluted whole blood in contact with the T-STHs were performed to assess the hemocompatibility of the formulations disclosed herein. As seen in FIG. 14, no significant differences were observed between STH formulations and were comparable to previously reported values for other engineered hemostats.

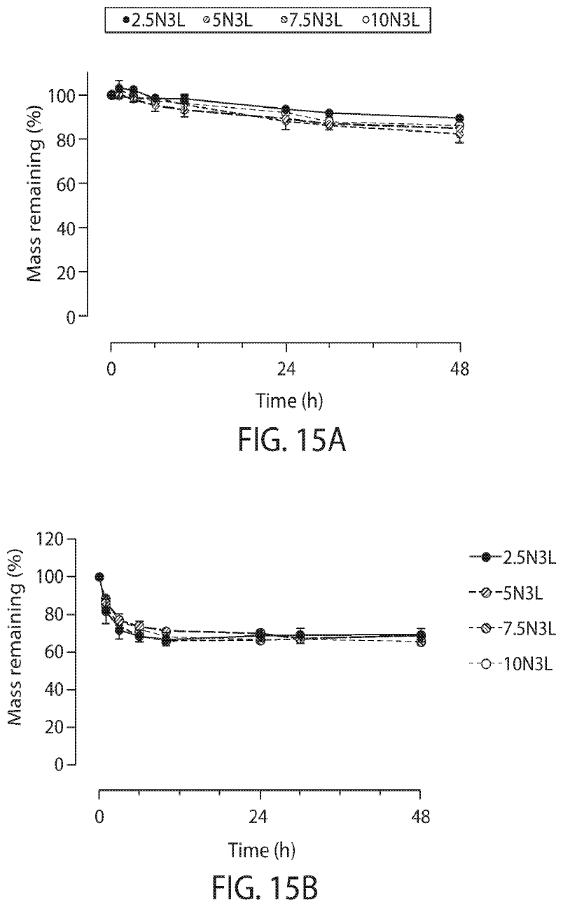

The short-term degradation of the T-STHs were also investigated over 48 hours under physiological conditions. A slow degradation rate was observed in PBS with approximately only 15% of mass loss (FIG. 15). However, in human plasma, a faster degradation was noted in the first 10 hours (˜35%) and little to no mass loss was observed for the remainder of the experiment (FIG. 5D). It should be noted that there was no significant difference in the mass loss observed between all four T-STH formulations, suggesting that polyNIPAM concentration does not affect the degradation of the T-STH. It is possible that the observed mass loss results from the T-STHs being held together by weak physical and electrostatic interactions between polyNIPAM and Laponite.

### 2.3. T-STH Prevents Blood Loss in an Ex Vivo Bleeding Model

The efficacy of the T-STHs disclosed herein was evaluated using an ex vivo bleeding model. Human blood vessel diameters range from 8 μm in capillaries to 25 mm for the aorta, with the blood flow rate in arteries and veins ranging from 3-26 mL/min and 1.2-4.8 mL/min, respectively. For the ex vivo studies, a syringe pump was used to flow human blood through medical-grade Tygon® tubing with an inner diameter of ˜2.4 mm at either 1 mL/min or 5 mL/min (FIG. 6A and FIG. 6B). An “injury” was created by puncturing the tubing carrying blood with a 1.5 mm biopsy punch, and the amount of blood loss from the injury site was weighed after 5 mins. For the untreated control, at the 1 mL/min blood flow rate, approximately 1300 mg of blood loss was observed. Not surprisingly, when the flow rate was increased to 5 mL/min, four times increase in blood loss (˜5200 mg) was observed.

In comparison, when treated with 1 mL of 10N3L, a significant decrease in blood loss from the “injury” site was observed for both 1 mL/min (p<0.05) and 5 mL/min (p<0.01) flow rates (FIG. 6C and FIG. 6D). At first, the T-STH creates a physical barrier at the “injury” site, which prevents blood loss. Over time, in response to a change in temperature, witnessed by a change in appearance from clear to white (FIG. 6C), 10N3L forms a plug that prevents further blood loss at the “injury” site and prevents the tube from “bleeding out.”

### 2.4. T-STHs Delivers Therapeutic Doses of DOXC In Vitro

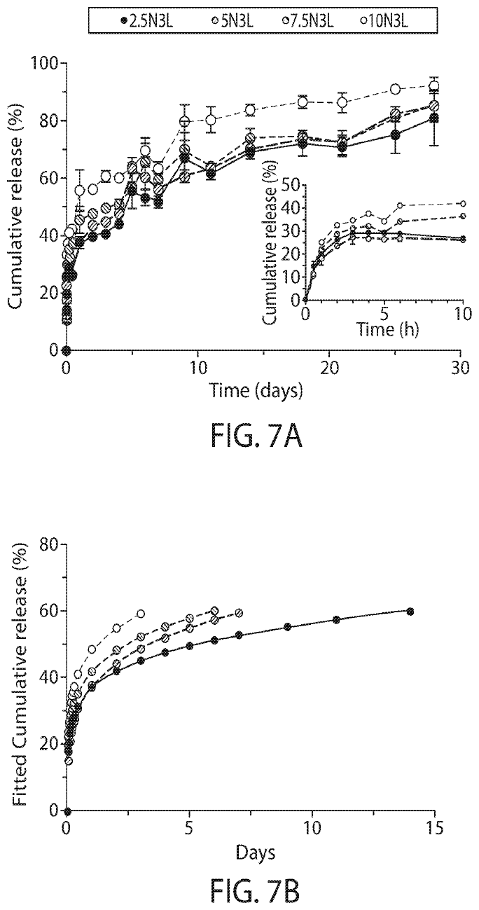

In battlefield injury and medical emergency situations, there is often a time delay between injury getting the victim to a medical facility for treatment. During this time, there is an increased chance of infection, which is reported to be the second leading cause of death following hemorrhage. Therefore, in addition to controlling bleeding, there is a need to prevent sepsis post-injury. To this end, the T-STH hemostats were loaded with DOXC, a broad-spectrum antibiotic, and its effectiveness in preventing gram-positive and gram-negative bacterial infections in vitro was investigated. Since DOXC is a positively charged hydrophilic molecule, the drug was loaded into the T-STH and formed a homogenous hydrogel during the physical mixing step. DOXC loaded T-STHs were stored in a refrigerator and protected from light until use. A 28-day drug delivery experiment using all the T-STH compositions disclosed herein was performed to understand the effect of polyNIPAM concentration on DOXC drug delivery release profile and kinetics. As seen in FIG. 7A, a burst release of approximately 25-40% cumulative release in the first 6 hours was observed. Higher polyNIPAM concentration resulted in a higher cumulative release. On day 28, for 10N3L, ˜92% cumulative release of the drug was observed compared to ˜85% cumulative release for 5N3L.

With the aid of the DDSolver add-ins plugin for MS Excel, the drug release profile of DOXC was modeled from the DOXC-loaded T-STHs. As seen in FIG. 7B and Table 1, the DOXC drug release profile from the T-STHs follows a Korsmeyer-Peppas model with R² greater than 0.97 and release exponent n<0.5 for all T-STH formulations, indicating a diffusion-controlled drug delivery mechanism.

TABLE 1
Korsmeyer-Peppas fit constants and R² values of DOXC drug
release from DOXC-loaded polyNIPAM and Laponite STH.

| T-STH compositions | Kₘ | n | R² |
|---|---|---|---|
| 2.5N3L | 20.585 | 0.183 | 0.978 |
| 5N3L | 18.691 | 0.208 | 0.987 |
| 7.5N3L | 22.670 | 0.191 | 0.977 |
| 10N3L | 25.216 | 0.227 | 0.971 |

This delivery profile is expected of polymeric systems and accounts for diffusion of water into the matrix, followed by swelling and eventual dissolution of the matrix. Specifically, for the T-STHs disclosed herein, at body temperature (above the LCST), the hydrophobic chains collapse and expel the water-soluble DOXC from the matrix. It should also be noted that DOXC is a positively charged molecule and would electrostatically interact with the negatively charged Laponite, which would slow its release from the hydrogel. As a result, a burst release was observed from the T-STHs followed by a more controlled release for the first two weeks, which plateaued out thereafter.

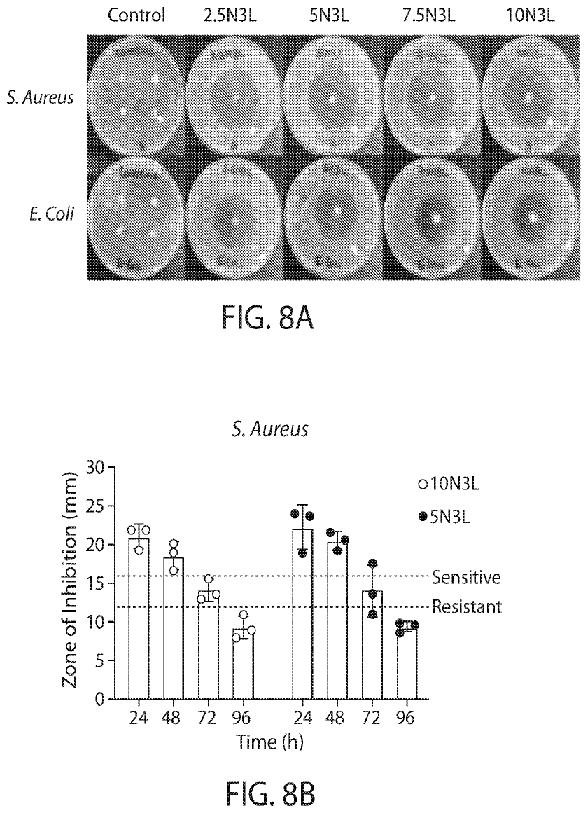

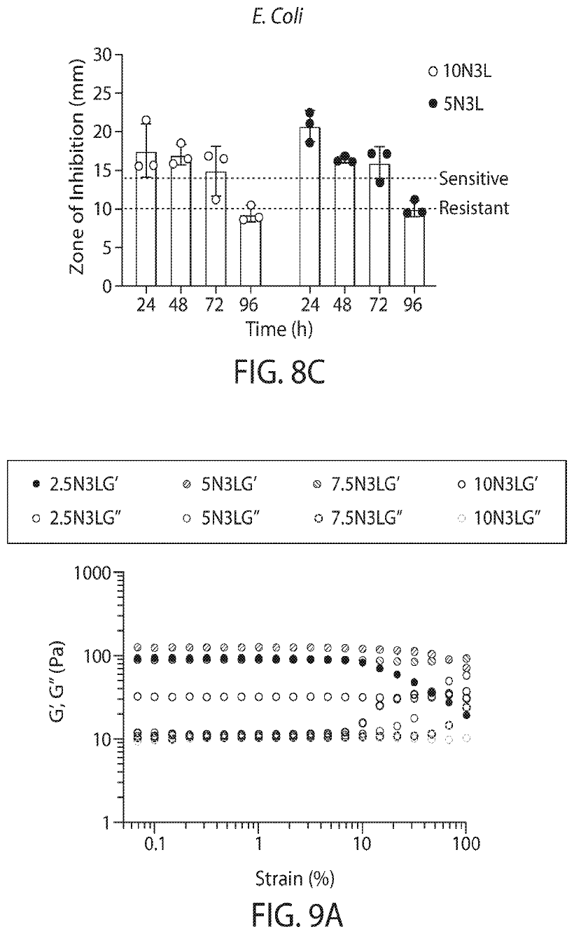

Finally, to investigate the activity of the released DOXC against common bacteria, ZOI assays were performed using S. aureus (gram-positive) and E. coli (gram-negative). Representative images of the inhibition zones after 24 h can be seen in FIG. 8A for all T-STHs. It has been reported that a 30 μg DOXC disk standard is effective against S. aureus and E. coli with a ZOI diameter ≥16 mm and >14 mm respectively, and is ineffective against them with a ZOI diameter ≤12 mm and ≤10 mm 46 (see Rahayu, I.; Widowati, R., Phytochemical, Antibacterial and Antioxidant Activities Test of Three Macro-Algae Phacophyceae Extracts from Pulau Tidung Coastal Kepulauan Seribu. 2020, 1, 1-26.). Based upon these standards, it was demonstrated that both 5N3L and 10N3L deliver effective doses of DOXC to inhibit S. aureus and E. coli infections for 72 h (FIG. 8B and FIG. 8C). The DOXC drug delivery studies and the ZOI assays show that the T-STHs disclosed herein are an effective vehicle for the short-term delivery of therapeutic doses of antibiotics.

Taken together, these results indicate that the polyNIPAM-Laponite based T-STH disclosed herein can function as an injectable hemostat while also delivering antibiotics. Currently, this injectable T-STH technology would be beneficial in puncture and penetrating wounds such as cuts, stabs, and gunshot wounds that lead to external hemorrhages. Furthermore, the T-STHs disclosed herein would also be useful in treating external hemorrhages that have debris, such as bullets and shrapnel, which have been lodged into the wound site due to the traumatic injury. Moreover, with the added ability of the T-STHs disclosed herein to deliver antibiotics, potential infections can be prevented while also stabilizing the hemorrhage and may be effective in preventing deaths in soldiers and victims as a result of both trauma-related hemorrhage and sepsis.

### 3. Conclusions

The polyNIPAM and Laponite combined T-STHs disclosed herein form injectable biomaterials that promote temperature-dependent in vitro coagulation. Moreover, these T-STHs can be loaded with a hydrophilic broad-spectrum antibiotic and deliver effective doses over several days. Of all the T-STH compositions disclosed herein, 10N3L exhibited improved coagulation in vitro, significantly reduced blood loss in an ex vivo model, and locally delivered therapeutic doses of DOXC for 72 h. Due to these unique features, the T-STHs disclosed herein can be used as an injectable hemostat to treat external hemorrhages and deliver antibiotics to prevent infections.

### Example 2

### 2.1. Abstract

Hemorrhage is the leading cause of death following battlefield injuries. Although several hemostats are commercially available, they do not meet all the necessary requirements to stop bleeding in combat injuries. Here, thermoresponsive shear-thinning hydrogels (T-STH) composed of a thermoresponsive polymer, poly(N-isopropyl acrylamide) (p(NIPAM)), and hemostatic silicate nanodisks, LAPONITE®, are engineered as minimally invasive injectable hemostatic agents. T-STH are physiologically stable hydrogels that can be easily injected through a syringe and needle and exhibits rapid mechanical recovery. Additionally, it demonstrates temperature-dependent blood coagulation owing to the phase transition of p(NIPAM). It decreases in vitro blood clotting times over 50% at physiological temperatures compared to room temperature. Furthermore, it significantly prevents blood loss in an ex vivo bleeding model at different blood flow rates (1 mL min⁻¹ and 5 mL min⁻¹) by forming a wound plug. More importantly, T-STH is comparable to a commercially available hemostat, Floseal, in terms of blood loss and blood clotting time in an in vivo rat liver bleeding model. Furthermore, once the hemorrhage is stabilized. T-STH can be easily removed using a cold saline wash without any rebleeding or leaving any residues. Taken together, these data suggest T-STH can be used as a first aid hemostat to treat external hemorrhages in emergency situations.

### 2.2 Introduction

Most battlefield injury-related mortalities occur in the premedical treatment facility environment before the injured soldier or victim can reach a hospital or a surgeon. Of these, hemorrhage is responsible for most trauma-related mortality and is the leading cause of death on the battlefield. Since 2008, QuikClot Combat Gauze® has been the gold standard for combat casualties by all US military branches. It is a gauze made of rayon and polyester blend loaded with kaolin, which helps control and stop bleeding by activating factor XII. This product, however, cannot be used in noncompressible hemorrhages or internal bleeding.

Polymeric hydrogels have commonly been used for hemostasis and wound healing applications. A variety of hemostats in the form of hydrogels, sheets, sponges, and powders have also been reported. Hydrogel-based hemostats, specifically have an added advantage due to their injectability and flowability which allows them to be used for fast and quick hemostasis from irregular-shaped wounds as well as intracavity injuries. However, among them, very few hemostats meet the requirements for clinical translation. Therefore, there is a need for innovative multifactorial hemostats that can be used in an external wound via simple injection. An ideal hemostat for battlefield injuries and pre-medical treatment facility hemorrhage control should have the following characteristics: (1) quick and adequate hemostasis in a wide range of injuries and wounds, (2) sustained hemostasis for several hours in situations of delayed evacuation, (3) easy removal without leaving any residues in the injury or wound, (4) ready to use and easy administration by a layperson with little to no training, (5) easy to manufacture and sterilize with low costs. (6) easily stored with prolonged stability even under extreme climate conditions, and (7) good biocompatibility with no adverse effects.

Shear-thinning hydrogels (STHs) can satisfy most of these requirements and have been developed using various materials. STHs are engineered such that their viscosity reduces under high shear stress making these materials deform easily through syringes, needles, and catheters and rapidly retain their original form after removing the mechanical force. Various labs have previously explored gelatin and LAPONITE®-based STHs for hemorrhage control and endovascular embolization. LAPONITE® (also sometimes referred to as silicate nanoplatelets) are highly charged nano-disks and have been shown to induce blood coagulation by interacting with platelets and concentrating clotting factors onto its surface.

Thermoresponsive polymers, such as poly(N-isopropyl-acrylamide) or p(NIPAM), above their lower critical solution temperature (LCST), transit from a soluble hydrophilic state to an insoluble hydrophobic state which makes them useful for drug delivery applications. Furthermore, the polymer back-bone can be modified with different polymers to tune the physical, mechanical, and drug release profile from these “smart” materials. Several research groups have also explored thermoresponsive STHs for use in drug delivery, 3D printing, as well as a range of other medical applications.

The motivation for this example is to demonstrate a short-term reversible hemostat, one that could prevent external bleeding and stabilize the patient in emergency situations but could also be easily removed without leaving any residue once the patient was transported to a medical treatment facility. Additionally, it should be mechanically stable under physiological conditions, accelerate local hemostasis, and can be easily removed once the patient has been stabilized. Here, an injectable hydrogel hemostat was engineered to function as a hemostatic plug at physiological conditions (FIG. 1). To do this, LAPONITE®'s ability to aid in coagulation was combined with the thermoresponsive nature of p(NIPAM) to create an injectable thermoresponsive STH or T-STH hemostat. Without being bound by theory, it is believed that the thermoresponsive nature of T-STHs disclosed herein plays a critical role in aiding blood coagulation. Once the hemorrhage has been stabilized, being thermoresponsive the hemostat can be easily washed away using a cold saline wash to remove any debris from the wound, such as bullets and shrapnel. To this end, the effect of varying p(NIPAM) concentrations on the rheological properties and injectability of the T-STHs was evaluated. Additionally, the thermo-responsive nature of T-STHs and their ability to function as a hemostat using in vitro clotting studies and an ex vivo bleeding model was assessed. Finally, the utility of injectable T-STH solutions as hemostats for minimally invasive treatments of hemorrhages using a rat liver bleeding model was demonstrated.

### 2.3. Materials and Methods

### 2.3.1. Materials

For the preparation of T-STHs, p(NIPAM) (MW 40 000 Da) was purchased from Sigma Aldrich (Cat #535311), and silicate nanoplatelets or LAPONITE®® XLG was purchased from BYK Additives Ltd. Citrated human whole blood was purchased from ZenBio (SER-WB). The blood type was O+ for the hemocompatibility studies and belonged to a 24-year-old African American male with a 35.7 BMI. For the ex vivo studies, the blood type was also O+ and belonged to a 53-year-old Hispanic male with a 25.68 BMI. In addition, calcium chloride (CaCl2) solution (0.1 M) and 0.9% saline solution was purchased from Spectrum Chemical Manufacturing Corp (C-092) and Teknova (S5815), respectively.

### 2.3.2. Preparation of Thermoresponsive STH (T-STH) Formulations and SEM Imaging

Stock solutions of p(NIPAM) (20 w/v %) and LAPONITE® (12 w/v %) were prepared in Milli-Q water (4° C.). Four different T-STH formulations (Table 2) were prepared by vortexing appropriate ratios of the p(NIPAM) stock, LAPONITE® stock, and Milli-Q water. The T-STH formulations had a general formula xNyL, where x=2.5, 5, 7.5 and 10 w/v % of p(NIPAM), and y=3 w/v % of LAPONITE®. The vortexing was done 3 times, at 3000 rpm for 5 min using a SpeedMixer™ (DAC 150.1 FVZ), and the T-STHs were stored overnight at 4° C. before use.

TABLE 2

| | PolyNIPAM | Laponite | Total solid |
|---|---|---|---|
| T-STH | (w/v %) | (w/v %) | (w/v %) |
| 2.5N3L | 2.5 | 3 | 5.5 |
| 5N3L | 5 | 3 | 8 |
| 7.5N3L | 7.5 | 3 | 10.5 |
| 10N3L | 10 | 3 | 13 |

The temperature-dependent phase transition of the T-STH was observed by taking digital images of the samples at room temperature and at 37° C. The morphology of dried T-STH with various compositions was characterized by scanning electron microscopy (SEM) (FEI Quanta 200, Hillsboro, OR). All samples for SEM imaging were kept at −80° C. for 24 h, freeze-dried for 48 h, and mounted onto a metallic stub using double-sided conductive carbon tape. Samples were then sputter-coated in an Ar atmosphere with an Au—Pd target at a peak current of 15 uA for 5 min and subsequently imaged using an accelerating voltage of 15-25 kV.

### 2.3.3. Rheological Analysis and Injectability of T-STH

T-STH shear rate, frequency sweeps, and recoverability were analyzed according to previously reported protocols with minor modifications. An Anton Paar MCR 302 rheometer was used for mechanical testing, and the data was recorded via Anton Paar Rheocompass software. Shear stress, viscosity, and storage moduli were measured with a 25 mm diameter parallel plate geometry with a rough surface, and a gap height of 500 μm was used. Mineral oil was added around the plate to prevent water evaporation from the T-STH once the sample was loaded. All T-STHs were equilibrated for 5 min before testing, followed by a 2 min steady shear at 10 s⁻¹. Steady shear rate sweeps investigated the shear-thinning properties of the samples at 25 and 37° C., and the viscosities of the materials were measured as a function of shear rate. Step-rate time-sweep was performed to investigate the thixotropic recovery properties of the samples between low shear strain (1%) and high shear strain (100%) at 25 and 37° C. Oscillation amplitude sweep and frequency sweeps were applied to measure the storage modulus (G′) and loss modulus (G″) of the samples at 37° C. Temperature sweeps from 15 to 45° C. at a heating rate of 1° C. min-1 were carried out to measure gelation temperature, whereas time sweeps at 37° C. were performed to investigate the gelation kinetics.

The injectability of T-STHs was analyzed using an Instron (Model 5542). Briefly, the T-STH was added to a 3 mL syringe and centrifuged at 2000 rpm for 5 min to pack the T-STH within each syringe. The T-STHs were then injected through the syringe either with no needle or a 23 G blunt needle (BD biosciences) using standard Luer-lock fittings. The syringe plunger was depressed using an upper compressive platen. The housing of the syringe or needle was fitted into a lower tensile grip to prevent movement during the experiment. An injection rate of 33.33 mL min⁻¹ was used for these tests, and the force on the plunger was measured with a 100 N load cell and recorded using Bluchill version 3 software. The plateau's average injection force (N) was obtained by quintuple measurements of three identical compositions of each T-STH formulation.

### 2.3.4. Degradation of T-STH

T-STH degradation was performed either in PBS or human plasma. First, human plasma was separated from citrated human whole blood by centrifugation at 3000 rpm for 15 min (Beckman Coulter Allegra™ 6R centrifuge) and stored at −80° C. until use. Next, 0.2 mL of each T-STH formulation was injected into 1.5 mL Eppendorf tubes, centrifuged to flatten, and weighed (˜175-200 mg). Then, 1 mL of pre-warmed PBS or human plasma was added to each sample and placed in a benchtop orbital shaker at 37° C. with constant shaking at 100 rpm (Barnstead Lab-Line MaxQ 4000). After incubation for 1, 3, 6, 10, 24, 30, and 48 h, the PBS or human plasma was removed, and the remaining T-STH was weighed. Each sample was replaced with either fresh PBS or human plasma and returned to the incubator. The relative weight percentage of each T-STH was defined as W %=(Wr/W0)×100, where Wr is the weight of the remaining T-STH at various time points, and W0 is the weight of T-STH at the initial state. T-STH degradation studies in both PBS and human plasma were performed in triplicates, and mass remaining (%) is reported as mean±standard deviation of the replicates.

### 2.3.5. Cytotoxicity and Hemocompatibility Assessment Studies

NIH/3T3 cells (ATCC, CRL 1658) were cultured in Dulbecco's modified Eagle's medium (Gibco, 1165092) and supplemented with 10% heat inactivated fetal bovine serum (Gibco™), 50 μg mL⁻¹ streptomycin, and 50 U mL-1 penicillin in 5% CO₂ at 37° C. Cells were seeded in a 24-well plate (1×10+ cells per well) and grown for 24 h with 1 mL of complete growth media. Next, 0.2 mL of T-STH was injected into transwell inserts (Costar, 3396) and sterilized via UV sterilization for 30 min. The eluates from the T-STH samples in the transwell inserts were then transferred to the 24-well plate with NIH 3T3 cells, and fresh complete growth media was added (1 mL) to the T-STH samples in the transwell inserts and changed daily throughout the experiment. Cytotoxicity was assessed at days 1, 3, and 5 using PrestoBlue™ cell viability reagent (A13261. ThermoFisher) following the manufacturer's protocol. Transwell inserts without any T-STH were used as controls. All data were normalized to the controls and reported as mean±standard error means of all replicates.

Hemolysis testing was performed according to previously published protocols. Citrated human whole blood was diluted 50× with 0.9% (w/v) saline solution. First, 0.1 mL of T-STH were injected into 1.5 mL Eppendorf tubes and briefly centrifuged to evenly flatten each sample. Next, 1 mL of diluted blood was added to each tube and incubated at 37° C. under agitation (100 rpm). After 2 h, the Eppendorf tubes were centrifuged at 14 000 rpm for 10 min, the supernatants were transferred into wells of a 96-well plate, and the absorbance of the supernatants was read at 542 nm. Saline and 2% SDS were used as negative and positive controls, respectively. Percent hemolysis was defined as H %=[(Asample−Aneg)/Apos]×100, where Asample is the absorbance at 542 nm of the T-STH-containing supernatant, Aneg is the absorbance of the saline-diluted blood, and Apos is the absorbance of the DI water-diluted blood. These hemolysis studies were performed in tri-plicates, and the hemolysis ratio (%) is reported as mean±standard deviation of all replicates.

### 2.3.6. In Vitro Blood Clotting Studies

Clotting times were measured according to previously reported protocols with minor variations. A volume of 630 μL of citrated whole blood was pipetted into a 1.5 mL Eppendorf tube, and a volume of 70 μL of 0.1 M CaCl₂) was then added, followed by vortexing for 10 s, to reactivate the blood. Immediately, 0.2 mL of the blood was transferred to 48-well plates. At every minute between 3 and 12 min, each well was washed with 0.9% saline solution to halt clotting. The liquid was immediately aspirated, and the samples were washed repeatedly until the wash solution was clear, indicating complete removal of blood components. For the T-STH samples, 0.2 mL of T-STH was injected into wells of a 48-well plate and centrifuged at 2000 rpm for 10 min to evenly flatten each sample. The final clotting time was determined when a uniform clot was formed, with no change in clot size in subsequent wells. These clotting time studies were performed at room temperature (˜25° C.) and 37° C. in triplicates for each time point.

Thrombus weights were determined according to previously published protocols with minor variations. Briefly, 0.1 mL of either 5N3L or 10N3L were injected into 1.5 mL Eppendorf tubes, followed by a short centrifuge cycle to flatten out each sample, and the tubes containing the T-STHs were weighed. Next, citrated human whole blood was reactivated with 0.1 M CaCl2 at a 9:1 ratio and vortexed for 10 s. Immediately, 0.1 mL of the blood was pipetted into the tubes containing the T-STH, and the tubes were transferred to an Eppendorf ThermoMixer® C maintained at 37° C. under constant agitation at 300 rpm. At each time point, clotting was stopped by adding 1 mL of 0.9% saline solution, followed by saline washes, until the wash solution was clear. Finally, the Eppendorf tubes with the newly formed clots were reweighed to determine the thrombus weights produced in the tubes. These studies were performed in triplicates for each time point, and the weight of the thrombus (mg) is reported as the mean±standard deviation of all replicates.

### 2.3.7. In Vitro Platelet Adhesion Studies

Quantification of platelet adhesion to T-STH gels was performed using previously published protocols. To obtain platelet-rich plasma (PRP), fresh citrated whole blood was centrifuged at 95 g for 15 min at 8° C. The transparent layer or PRP was transferred to another vial and the RBC layer was discarded. 200 μL of PRP was dispensed onto the dried T-STH samples and incubated at 37° C. for 1 h. After incubation, the samples were gently rinsed three times with PBS, and the adherent platelets were fixed with 4 v/v % paraformaldehyde (in PBS) overnight at 4° C. After fixation, the samples were rinsed in PBS (three times) and serial dehydrated with 10%, 25%, 50%, 75%, 90%, and 100% ethanol for 10 min each. The dehydrated samples were mounted onto a metallic stub using double-sided conductive carbon tape. Samples were then sputter-coated in an Ar atmosphere with an Au—Pd target at a peak current of 15 μA for 5 min and subsequently imaged with a ZEISS Supra 40VP SEM using an accelerating voltage of 12 kV. The platelet adhesion results were interpreted qualitatively by observing the SEM images.

### 2.3.8. Ex Vivo Bleeding Model

The ex vivo bleeding model setup had two sections. The “flow” section comprised a syringe pump (Braintree Scientific, Model BS8000) housed in an incubator to maintain the blood temperature at 37° C. Medical grade tubing was used as artificial blood vessels (Tygon® tubing ND-100-65; inner diameter 3/32 inches and outer diameter 5/32 inches) to flow blood from the “flow” section to the “injury” section, which was outside the incubator but placed on a heating pad (VIVOSUN reptile heat mat and digital thermostat combo) set to 37° C. and a portable heater (AIR KING non-oscillating portable electric heater) was used to maintain the ambient temperature at 37° C. An image of the ex vivo bleeding model setup can be found in FIGS. 6A and 6B.

Briefly, 0.1 M CaCl₂) solution was mixed with citrated whole blood at a 1:9 ratio, and vortexed for 10 s. The activated blood was gently transferred to a syringe and was secured in the syringe pump. A 23 G needle with Tygon® tubing was then attached to the syringe. To mimic various blood flow rates in the body, these experiments were performed at two different flow rates: 1 mL min-1 and 5 mL min-1. Once the blood started flowing at the end of the tube, an “injury” was created approximately 10 inches from the tubing end using a 1.5 mm biopsy punch to puncture the tube. The “injury” was either left untreated (control) or 1 mL of 10N3L was applied to the injury site. At every minute, from t=0 min to 5 min, a digital image was taken, and the amount of blood loss (mg) was measured by weighing the Whatman filter paper at the end of the experiment (5 min). The ex vivo studies were performed in triplicates, and the amount of blood loss (mg) is reported as the mean±standard deviation of all replicates.

### 2.3.9. In Vivo Rat Liver Bleeding Model

15 male rats were purchased from Charles River and housed in an animal facility (Lundquist Institute, Torrance, CA) at a con-trolled temperature of 23±1° C., and humidity of 55±5%, in a 14 h light/10 h dark cycle for one week before the animal procedure. All the animal experiments were performed with the approval of the Institutional Animal Ethics Committee of the Lundquist Institute (IACUC number: 32705-01), where the study was executed following the guidelines of the Committee for Control and Supervision of Experimental Animals, USA. On the day of surgery, all animals were randomly divided into 3 groups (n=5): sham control, commercially available Floseal hemostatic matrix (Baxter), and the T-STH composed of 10N3L. All the equipment were sterilized and dried in a sterile environment before the procedure. The animals were anesthetized using isoflurane inhalation (1-4%), and under anesthesia, the surgery site was shaved and sterilized with betadine, and an incision was made horizontally to expose the liver. The left middle lobe of the liver was lifted upward, placed on a Whatman weighing paper, and a standardized liver wound (4 mm) was created with a disposable surgical biopsy punch at the base of the lobe. After wound bleeding, the speed and initial amount of bleeding were qualified for subsequent hemostatic experiments. The animals were then treated with different hemostatic agents on the liver wound to stop the bleeding. For example, the sham control group, underwent active bleeding without any hemostatic treatment. In the second and third group, the bleeding liver was treated with commercially avail-able Floseal (0.1 mL) and 10N3L (0.1 mL), respectively, on the bleeding site. The clotting time after applying the experimental material (control and hemostats) and the amount of blood loss was measured by weighing the Whatman paper.

### 2.3.10. Graphing and Statistical Analysis

GraphPad Prism 9 software was used for graphing and plotting data for this research, as well as for performing statistical analysis. When appropriate a one-way ANOVA analysis, followed by a Tukey's post hoc analysis, was used to determine statistical differences between treatment groups. For the cytotoxicity studies, a two-way ANOVA analysis was performed to determine statistical differences between treatment groups and between days. For the ex vivo bleeding studies, an unpaired Student's t-test was used to observe any statistical difference in blood loss between the control and 10N3L treatment group. In all cases, a was set to 0.05.

### 2.4. Results and Discussion

### 2.4.1. T-STHs are Thermoresponsive, Injectable, and Non-Cytotoxic

Shear-thinning injectable materials have a lot of potential for developing minimally invasive therapies. LAPONITE® in its hydrated state exhibits shear-thinning properties. Over the past several years, several labs have developed shear-thinning hydrogels from porcine gelatin and LAPONITE® as embolic agents and drug delivery systems for the treatment hepatocellular carcinoma. Inspired by previous research, in this study, a thermoresponsive hydrogels composed of p(NIPAM) and LAPONITE® was engineered. The thermoresponsive nature of p(NIPAM) was utilized to develop a reversible hemostat that would harden and form a wound plug at body temperature but could also be easily removed with a cold saline wash. Stock solution of p(NIPAM) and LAPONITE® were homogenized via vigorous agitation using a speed mixer to prevent clumping of the LAPONITE® during gelation. According to previous research on gelatin-LAPONITE®-based STHs, LAPONITE® concentration of 3% w/w resulted in stable T-STHs that were effective in clotting blood for endovascular embolization. For the fabrication of T-STH formulations, the LAPONITE® concentration was kept constant at 3 w/v %, while the p(NIPAM) concentration was varied from 2.5 w/v % to 10 w/v % (Table 2).

The resulting T-STHs had a clear appearance at room temperature. Increasing p(NIPAM) concentration resulted in hydrogels that were more gelatinous in appearance. At 37° C., the clear appearance changes to an opaque and white hydrogel (FIG. 2A-2B). This is a result of the phase transition of p(NIPAM) from a hydrophilic state below its LCST to a hydrophobic state above its LCST. In addition, the microstructure of T-STH was observed using SEM after freeze-drying (FIG. 2C). It appears that increasing p(NIPAM) concentration within the T-STH results in hydrogels with lower porosity. However, it should be noted that the actual porosity and pore sizes of the T-STH were not measured in this study and will need to be further investigated.

The rheological properties of T-STHs were investigated using a rotational rheometer at both room temperature (25° C.) and body temperature (37° C.). T-STH formulations showed a slightly broader linear viscoelastic range (LVER) at 25° C. as compared to 37° C., and the strain required to break the hydrogel network structure was greater than 10% at both temperatures. It should be noted that increasing the p(NIPAM) concentration in the T-STHs resulted in stronger gels at 25° C. (FIG. 9A). However, at 37° C., a larger p(NIPAM) concentration resulted in slightly weaker gels (FIG. 9B). Without being bound by any particular theory, it is believed that at temperatures above the LCST, p(NIPAM) becomes more hydrophobic and interacts with its own polymer chains rather than the highly charged LAPONITE®, resulting in weaker gels. Additionally, the frequency-dependent rheology of T-STHs were acquired in the LVER. For all samples, the G′ exceeds the G″ values at both temperatures, indicating the formation of hydrogels. At 25° C., the G′ value of the T-STHs decreases with p(NIPAM) concentration, whereas, at 37° C., the opposite trend is observed, indicating that p(NIPAM) contributes to the mechanical strength of the T-STHs (FIGS. 10A and B). This is advantageous, as a higher G′ at physiological temperatures would result in a harder gel that could function as a wound plug and prevent bleeding from a hemorrhaging wound.

To further explore the thermoresponsive behavior of the T-STHs, the temperature-dependent changes in G′ and G″ values of T-STH formulation was investigated, either through a slow temperature ramp from 15° C. to 45° C. or a sudden temperature change from 25° C. to 37° C. (FIGS. 12A and B). The slow temperature ramp was carried out to observe the transition temperature of the T-STHs, while the sudden temperature change was performed to recapitulate the instance in which the T-STH would be applied to a bleeding patient (37° C.) from room temperature (25° C.). The data reveals that the G′ values are always higher than the G″ values, which indicates that the T-STH is always in a hydrogel state. However, during the slow temperature ramp, it was observed that the G′ and G″ values started to increase around 31-32° C. which is comparable to the LCST of p(NIPAM) (˜32° C.). This indicates that p(NIPAM) and LAPONITE® are held together by weak electrostatic interactions in the T-STHs. Furthermore, the results indicate that in response to a sudden temperature change from 25° C. to 37° C., increasing p(NIPAM) concentration results in an increase as well as a faster change in G′ and G″ values of T-STHs.

Next, the shear-thinning properties of T-STH formulations at 25° C. (FIGS. 3A and B) and 37° C. (FIGS. 3D and E) were studied. For both temperatures, it was observed that p(NIPAM) concentration did not significantly affect the viscosity of T-STH, except in the cases of 2.5N3L and 10N3L (p<0.01). However, a change in temperature from 25° C. to 37° C. resulted in significantly more viscous STHs for each formulation. Several cycles of high strain were also applied (100% oscillatory strain) to break the network structure of the T-STH formulations, followed by low strain (1% oscillatory strain) to monitor the recovery of the storage modulus (G′) of the T-STH at both room temperature and body temperature (FIGS. 3C and F). At 25° C., the strain had minimal effect on the G′ values of T-STH. However, at 37° C., the effect of strain was more pronounced. It should be noted, that at both temperatures, each of the T-STH formulations possess excellent thixotropic recovery properties.

To assess the case of injectability of T-STH, the injection process was replicated using a mechanical tester. The force required to inject T-STH from 3 mL syringes was measured either with or without a 23 G blunt needle (FIG. 4A). These experiments were only performed at room temperature. The force required to inject T-STH from a 3 ml syringe (without a needle) ranged between 1-2 N, with no significant differences between the T-STH formulations. However, with a 23 G blunt needle, the applied force increased with increasing p(NIPAM) concentration and ranged from ˜3.6 N for 2.5N3L to ˜10.6 N for 10N3L (FIG. 4C). It was observed that in all cases, the applied force increased linearly until it plateaued at the injection force (FIG. 4B). These injection forces are easily achieved manually and do not require additional equipment for the application, allowing these T-STHs to be easily implemented in emergency medical situations for application on external hemorrhaging wounds.

The short-term degradation of T-STHs was investigated over 48 h under physiological conditions. A slow degradation rate was observed in PBS with approximately only 15% of mass loss. However, in human plasma, a faster degradation was observed in the first 10 h (˜35%) and little to no mass loss for the remainder of the experiment. It should be noted that there was no significant difference in the mass loss between all four T-STH formulations, suggesting that p(NIPAM) concentration does not affect the degradation of the T-STH. Without wishing to be bound by any particular theory, it is believed that the observed mass loss results from T-STH being held together by weak physical and electrostatic interactions between p(NIPAM) and LAPONITE®. Since T-STH hemostats are meant to be used for short-term use until the victim reaches a medical treatment facility (<24 h), the observed T-STH degradation is a non-issue.

Next, it was shown that T-STHs are non-cytotoxic to NIH/3T3 fibroblasts over five days as determined by both PrestoBlue™ cell viability and LIVE/DEAD assays (FIGS. 13A and B). Furthermore, a 2-way ANOVA analysis of the normalized absorbance values determined no significant differences between the treatment groups or days. Moreover, hemolysis assays of diluted whole blood in contact with T-STHs were performed to assess the hemocompatibility of the formulations. FIG. 14 shows that no significant differences between STB formulations which were comparable to previously reported values for other engineered hemostats.

### 2.4.2. T-STHs Promote Temperature-Dependent Coagulation In Vitro

Based on the initial characterization of T-STH, which included rheological characterization, case of injectability, and cytotoxicity testing, it was decided to focus specifically on 5N3L and 10N3L for the blood coagulation experiments. The hemostatic ability of 5N3L and 10N3L was evaluated by monitoring the clotting time of whole blood in contact with the T-STH (FIG. 5A). The citrated whole blood (CWB) procured for these experiments coagulated in 11-12 min under physiological conditions (37° C.) after activation with CaCl2. Under the same conditions, similar clotting times were observed for 5 wt % p(NIPAM). Increasing the p(NIPAM) concentration lowered the clotting time to 8-9 min, indicating that p(NIPAM) concentration affects clotting time. It has been previously hypothesized that negatively charged LAPONITE® interacts with platelets and other clotting factors in whole blood, which aids in coagulation. Thus, incorporating LAPONITE® into T-STH formulations further reduced clotting time to ˜5.6 min for 10N3L and was comparable to 3 wt % LAPONITE® controls, which is over a 50% reduction in clotting time (FIG. 5B).

Interestingly, as seen in FIG. 5A, the clotting times for 5N3L and 10N3L at room temperature were comparable to activated CWB. A slight color change was observed early on, which may be attributed to LAPONITE®; however, coagulation only occurred around 12 min. Without wishing to be bound by any particular theory, it is believe that below the LCST of p(NIPAM) (˜32° C.), the polymer is in its soluble hydrophilic state and does not allow the blood to interact with LAPONITE®. However, above 32° C., as the p(NIPAM) changes to an insoluble hydrophobic form, it entraps the blood within the polymer matrix and allows the LAPONITE® to interact with the blood better. This elucidates that the thermoresponsive behavior of the hemostats aid in the coagulation of blood. Moreover, the thrombus weights were measured as a function of time for both 5N3L and 10N3L and their plots have a characteristic S-shaped curve with a clot starting to form around 3 min and plateaued out around 6-7 min (FIG. 5C), which correlated well with the clotting time data.

It is well known that platelets play an important role in the coagulation cascade. For a material to be hemostatic in nature it needs to exhibit strong platelet adhesion, activation and aggregation to the material surface. To demonstrate the hemostatic nature of T-STH hemostats, a platelet adhesion test was performed. After an hour of incubation of T-STH hemostats with PRP, a large number of platelets were adhered and aggregated to the surface of both 5N3L and 10N3L surfaces, as assessed via SEM. This is not surprising, since it has been previously reported that the highly negative charge of LAPONITE® leads to platelet adhesion and activation, and eventually triggers the coagulation cascade.

### 2.4.3. T-STH Prevents Blood Loss at Different Blood Flow Rates in an Ex Vivo Bleeding Model

Following the in vitro blood clotting studies, for the remainder studies, it was decided to focus on 10N3L to evaluate the efficacy of T-STH using an ex vivo bleeding model. Human blood vessel diameters range from 8 μm in capillaries to 25 mm for the aorta, with the blood flow rate in arteries and veins ranging from 3-26 mL min-1 and 1.2-4.8 mL min⁻¹. respectively. For ex vivo studies, a syringe pump was used to flow human blood through medical-grade Tygon® tubing with an inner diameter of ˜2.4 mm at either 1 mL min-1 or 5 mL min-1 (FIG. 6A). An “injury” was created by puncturing the tubing carrying blood with a 1.5 mm biopsy punch, and the amount of blood loss from the injury site was weighed after 5 min. For the untreated control, at the 1 mL min-1 blood flow rate, blood loss was approximately 1300 mg. Not surprisingly, when the flow rate was increased to 5 mL min-1, a four-fold increase in blood loss (˜5200 mg) was observed.

In comparison, when treated with 1 mL of 10N3L, a significant decrease in blood loss from the “injury” site for both 1 mL min⁻¹ (p<0.05) and 5 mL min⁻¹ (p<0.01) flow rates was observed (FIGS. 6C and D). At first. T-STH creates a physical barrier at the “injury” site, which prevents blood loss. Over time, in response to a change in temperature, witnessed by a change in appearance from clear to white (FIG. 6C), 10N3L forms a plug that prevents further blood loss at the “injury” site and prevents the tube from “bleeding out.” It should be noted that a portable heater was used to maintain the ambient temperature around 37° C. at the “injury” site. As a result, the phase changes observed in 10N3L during the ex vivo study may not correctly replicate the body temperature. Despite this shortcoming. T-STH still effectively prevent blood loss from an “injury.” The ex vivo results, combined with in vitro blood coagulation results, show that T-STHs are effective hemostats for bleeding wounds and can prevent blood loss from bleeding veins and small arteries.

### 2.4.4. T-STH Compares to Commercially Available Hemostats in an In Vivo Liver Bleeding Model

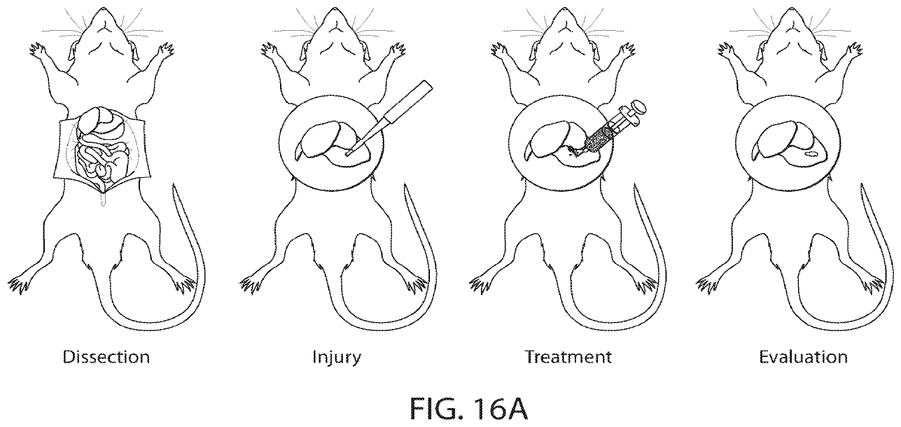

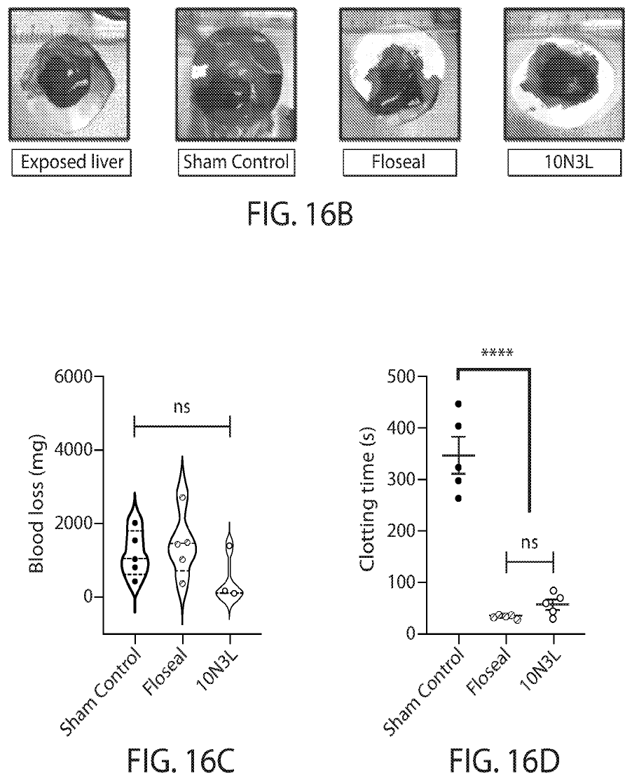

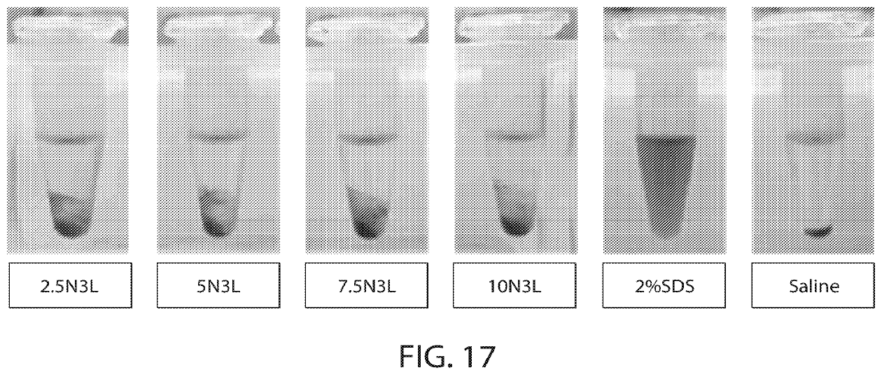

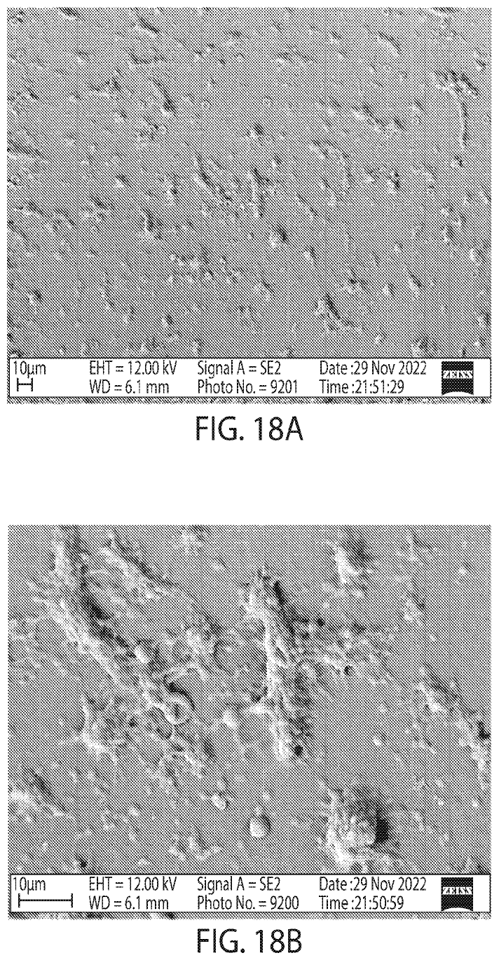

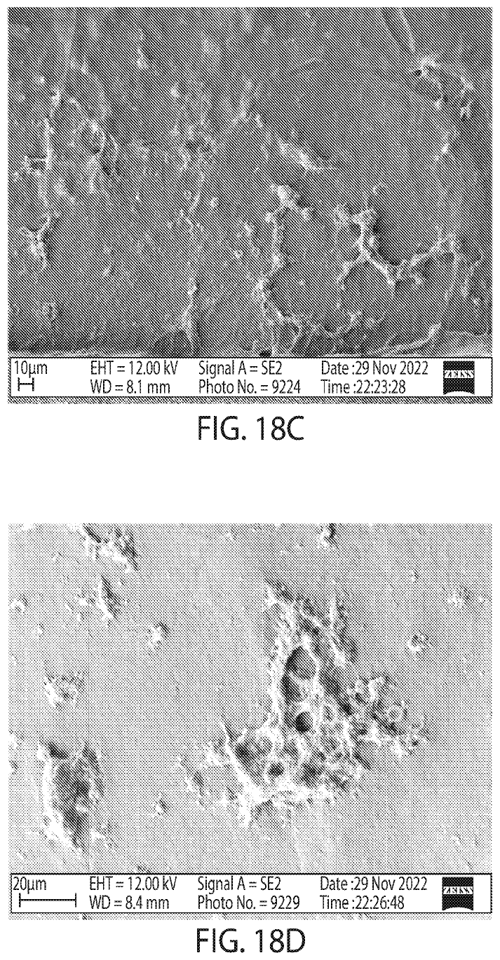

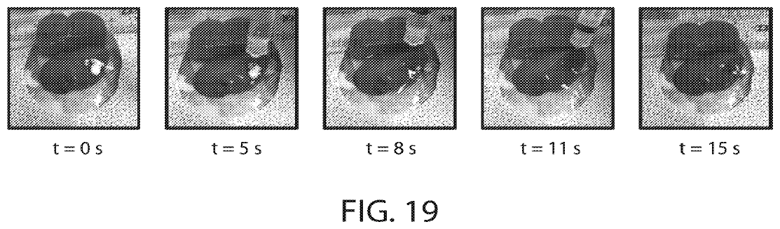

An in vivo rat liver bleeding model was used to validate the hemostatic performance of the newly developed p(NIPAM) and LAPONITE®-based T-STH. When 10N3L is compared against a commercially available hemostat, Floseal, an injectable hemostat matrix composed of gelatin and human thrombin. A schematic of the overall in vivo liver bleeding study can be found in FIG. 16A. Upon injury of the liver, blood loss and clotting times were used as determinants of effective hemostasis.

Representative images of the exposed liver (FIG. 16B) and amount of blood loss after treatment with or without hemostats can be found in FIG. 16B (e.g., Floseal). The blood loss in the sham control, Floseal, and 10N3L treatment groups were 1121±620 s, 1398±848 s, and 372±554 s respectively (FIG. 16C). The clotting time in the sham control group averaged 345±75 s in the absence of hemostatic material treatment. However, the Floseal (35±5 s) and 10N3L group (58±21 s) stopped bleeding quickly (FIG. 16D). Blood clotting times for the treatment groups decreased significantly compared to sham controls, and it is worth noting that there were no significant differences between 10N3L and Floseal (p=0.709). Therefore, treatment with 10N3L in the liver bleeding model resulted in lesser blood loss and comparable coagulation to Floseal, indicating that 10N3L contains functional components which could effectively improve hemorrhage control capability comparable to the speedy coagulation observed in Floseal which is attributed to thrombin. More importantly, these results demonstrate that T-STH achieved the ‘standard of care’ level for a commercially available hemostat in an in vivo bleeding model. Furthermore, the case of applicability and removal of hemostats was investigated by observing untreated wounds, treated wounds, and wounds after the materials were removed using a cold saline wash. Compared to Floseal, there were no 10N3L residues on the wounds after the saline wash. This is because as the temperature lowers below the LCST of p(NIPAM), 10N3L becomes more hydrophilic or hydrated and can be easily removed using a simple washing process.

As previously discussed, hemorrhage control in the premedical treatment facility is critical for the survival of a patient, which drives the development of hemostatic materials for the battlefield and emergency first aid. Ideal hemostatic agents are biocompatible, non-cytotoxic, and beneficial for wound closure. Additionally, they are inexpensive and simple to use for a wide range of injuries. Floseal contains human thrombin, which helps convert fibrinogen to fibrin, and upon injection forms a crosslinked hemostatic plug to prevent bleeding. In comparison to Floseal, T-STH demonstrated promising results without the use of clotting factors. T-STHs enables indirect hemostasis via phase transition of p(NIPAM) to create a stable hemostatic wound plug at physiological temperatures and prevents blood loss from bleeding wounds independent of the normal coagulation mechanism. Additionally, LAPONITE® interacts with platelets to form the soft platelet plug, which eventually turns into a strong fibrin clot. Synergistically, the phase transition of p(NIPAM) and the platelet interaction and activation by LAPONITE® helps to form the wound plug and staunch hemorrhage from a bleeding wound.

Moreover, as compared to existing hydrogel-based hemostats and commercially available hemostats like Floseal, p(NIPAM) and LAPONITE® based T-STHs are reversible. Once the hemorrhage has been stabilized and the patient is safely transported to a hospital, T-STHs can be easily removed from the wound using a cold saline wash and not leave any residues behind. Recently, Liang et al. have developed adhesive hydrogel sealants that can be easily removed post-wound healing via dissolution. Similarly, due to the thermoresponsive nature of the T-STH hemostats they can also be removed on demand after achieving hemostasis. In emergency and battlefield set-tings, when T-STH is applied to a bleeding wound it will quickly undergo a phase transition to form a wound plug and prevent bleeding. Once the bleeding has stopped and the patient has been safely transported to a medical treatment facility, the T-STH hemostat can be washed away with cold saline without leaving any residue, and the clinician can perform appropriate surgery and treatment for the patient. This ability of easy application and removal of T-STH hemostats provides first responders in emergency situations and clinicians in hospital settings with an easy, yet effective minimally invasive tool for the treatment of external hemorrhages.

### 2.5. Conclusions

p(NIPAM) and LAPONITE®-based T-STHs formed injectable biomaterials that could be easily administered via a syringe, had higher G′ values at physiological temperatures, were hemo- and cytocompatible, and promoted temperature-dependent in vitro coagulation. Of all T-STH compositions, 10N3L exhibited improved coagulation in vitro, significantly reduced blood loss in an ex vivo model from two different blood flow rates (1 and 5 mL min-1), and was comparable to a commercially available hemostat, Floseal, in an in vivo rat bleeding model in terms of blood loss and clotting time. Moreover, T-STHs are mechanically stable under physiological conditions, accelerate local hemostasis without any clotting factors, and can be easily removed using a cold saline wash without leaving any residues. Due to these unique features, it is believed that T-STHs would function well in battlefield and emergency situations as an injectable hemostat first aid to treat external hemorrhages.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teaching of the present disclosure is/are used. Those skilled in the art will recognize or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list. “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of.” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either.” “one of.” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B.” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An article, comprising:
a thermoresponsive polymer dissolved in solution; and
a plurality of nanoparticles suspended in the solution;
wherein the solution is thixotropic and comprises a lower critical solution temperature (LCST) of at least 25° C.

2. The article of claim 1, wherein the thermoresponsive polymer comprises a poly(acrylamide).

3. The article of claim 2, wherein the poly(acrylamide) comprises poly(N-isopropyl acrylamide).

4. The article of claim 2 or 3, wherein the poly(acrylamide) comprises poly(N,N-diethyl acrylamide).

5. The article of any one of claims 2-4, wherein the poly(acrylamide) comprises poly(N-ethylmethacrylamide).

6. The article of any one of claims 1-5, wherein the thermoresponsive polymer comprises a poly(caprolactam).

7. The article of claim 6, wherein the poly(caprolactam) comprises poly(N-vinyl caprolactam).

8. The article of any one of claims 1-7, wherein the thermoresponsive polymer comprises a poly(vinyl ether).

9. The article of claim 8, wherein the poly(vinyl ether) comprises a poly(methyl vinyl ether).

10. The article of any one of claim 8 or 9, wherein the poly(vinyl ether) comprises a poly(2-ethoxyethyl vinyl ether).

11. The article of any one of claims 1-10, wherein the thermoresponsive polymer is a homopolymer.

12. The article of any one of claims 1-11, wherein the thermoresponsive polymer is a block copolymer.

13. The article of any one of claims 1-12, wherein the thermoresponsive polymer is a branched polymer.

14. The article of any one of claims 1-13, wherein the thermoresponsive polymer is dissolved in the solution at a concentration of between 0.25 g/dL to 5 g/dL.

15. The article of any one of claims 1-14, wherein the plurality of nanoparticle comprises a synthetic smectic clay.

16. The article of any one of claims 1-15, wherein the plurality of nanoparticles comprises a ceramic nanoparticle.

17. The article of any one of claims 1-16, wherein the plurality of nanoparticles comprises a metal nanoparticle.

18. The article of any one of claims 1-17, wherein the plurality of nanoparticles comprises a polymeric nanoparticle.

19. The article of any one of claims 1-18, wherein the plurality of nanoparticles comprises a lipid nanoparticle.

20. The article of any one of claims 1-19, wherein the plurality of nanoparticles comprises a carbon-based nanoparticle.

21. The article of any one of claims 1-20, wherein the plurality of nanoparticles comprises Laponite.

22. The article of any one of claims 1-21, wherein the plurality of nanoparticles comprises a negatively charged outer surface.

23. The article of any one of claims 1-22, wherein the plurality of nanoparticles comprises a positively charged outer surface.

24. The article of any one of claims 1-23, wherein the plurality of nanoparticles comprises a coating.

25. The article of any one of claims 1-24, wherein the plurality of nanoparticles is present in the solution at a concentration of between 0.05 g/dL and 5 g/dL.

26. The article of any one of claims 1-25, wherein the plurality of nanoparticles degrade into nontoxic components.

27. The article of any one of claims 1-26, wherein the solution is thixotropic at a shear rate of between 0.1 1/s and 1.0 1/s.

28. The article of any one of claims 1-27, wherein the solution comprises a hydrogel between 32° C. and 37° C.

29. The article of claim 28, wherein the hydrogel has a storage modulus of between 10 and 1000 Pa.

30. The article of any one of claims 1-29, wherein the solution has a viscosity of between 1 and 100 Pa see at 25° C.

31. The article of any one of claims 1-30, wherein the solution has a viscosity of between 1 and 1000 Pa see at 37° C.

32. The article of any one of claims 1-31, wherein the solution requires an injection force of between 1 and 10 N.

33. The article of any one of claims 1-32, wherein the solution further comprises one or more antibiotics.

34. The article of any one of claims 1-33, wherein the solution further comprises one or more clotting factors.

35. The article of claim 34, wherein the one or more clotting factors comprises thrombin.

36. The article of any one of claim 34 or 35, wherein the one or more clotting factors comprises clotting factor XIII.

37. The article of any one of claims 34-36, wherein the one or more clotting factors comprises clotting factor VII.

38. The article of any one of claims 1-37, wherein the solution further comprises one or more antifibrinolytic agents.

39. The article of claim 38, wherein the one or more antifibrinolytic agents comprises tranexamic acid.

40. The article of any one of claim 38 or 39, wherein the one or more antifibrinolytic agents comprises epsilon-aminocaproic acid.

41. The article of any one of claims 1-40, wherein the article is a hemostatic agent.

42. An article, comprising:
a thermoresponsive polymer dissolved in a thixotropic solution, wherein the polymer is insoluble in the solution at between 32° C. and 37° C.; and
a plurality of charged particles suspended in the thixotropic solution.

43. A method, comprising:
injecting a liquid solution into a site of hemorrhage in a subject, wherein the liquid solution comprises a polymer and a plurality of nanoparticles, wherein upon injecting the liquid solution the polymer produces a hydrogel and the plurality of nanoparticles concentrate one or more clotting factors at the site of hemorrhage; and
forming a plug within the site of hemorrhage.

44. The method of claim 43, wherein the plug comprises the hydrogel.

45. The method of any one of claim 43 or 44, wherein forming the hydrogel uses the subject's body temperature.

46. The method of claim 45, wherein using the subjects body temperature causes the polymer to undergo a phase change from a liquid to a solid.

47. The method of any one of claims 43-46, wherein forming the hydrogel uses an external heat source.

48. The method of claim 47, wherein using the external heat source causes the polymer to undergo a phase change from a liquid to a solid.

49. The method of any one of claims 43-48, wherein the plug comprises at least a part of the plurality of nanoparticles.

50. The method of any one of claims 43-49, wherein the plurality of nanoparticles comprises a negatively charged outer surface.

51. The method of any one of claims 43-50, wherein the plurality of nanoparticles comprises a positively charged outer surface.

52. The method of any one of claims 43-51, wherein the plurality of nanoparticles comprises Laponite.

53. The method of any one of claims 43-52, wherein the plurality of nanoparticles binds to one or more clotting factors.

54. The method of any one of claims 43-53, wherein the plug comprises a blood clot.

55. The method of claim 54, wherein the blood clot has a maximum amplitude of between 55 mm to 80 mm.

56. The method of any one of claim 54 or 55, wherein the blood clot has a K value of between 1 min and 4 mins.

57. The method of any one of claims 54-56, wherein the blood clot has an R-value of between 4 min and 8 min.

58. The method of any one of claims 54-57, wherein the blood clot has an alpha-angle of between 47 degrees and 74 degrees.

59. The method of any one of claims 54-58, wherein the blood clot has an LY30% value of between 0% and 8% of the total mass of the blood clot.

60. The method of any one of claims 43-59, wherein the polymer is insoluble in the liquid solution at a temperature of between 32° C. and 37° C.

61. The method of any one of claims 43-60, wherein the polymer comprises a poly(acrylamide).

62. The method of any one of claims 43-61, wherein the polymer comprises a poly(caprolactam).

63. The method of any one of claims 43-62, wherein the polymer comprises a poly(vinyl ether).

64. A method, comprising:
administering, to a site of hemorrhage in a subject, a solution comprising a polymer and charged silicate nanoparticles, wherein upon administration, the solution is heated by blood at the site of hemorrhage to produce a hydrogel comprising the polymer and clotting proteins inter-dispersed with the charged silicate nanoparticles.

65. The method of claim 64, wherein the hydrogel has a maximum amplitude of between 55 mm to 80 mm.

66. The method of any one of claim 64 or 65, wherein the hydrogel has a K value of between 1 min and 4 mins.

67. The method of any one of claims 64-66, wherein the hydrogel has an R-value of between 4 min and 8 min.

68. The method of any one of claims 64-67, wherein the hydrogel has an alpha-angle of between 47 degrees and 74 degrees.

69. The method of anyone of claims 64-68, wherein the hydrogel has an LY30% value of between 0% and 8% of the total mass of the blood clot.

70. A thixotropic solution, comprising:
a polymer able to form a hydrogel at a temperature between 32° C. and 37° C.; and
charged lithium sodium magnesium silicate particles suspended in the thixotropic solution.

71. An article, comprising:
a solution comprising a polymer able to form a hydrogel at a temperature between 32° C. and 37° C., and charged lithium sodium magnesium silicate particles.

72. The article of claim 71, wherein the polymer comprises a poly(acrylamide).

73. The article of claim 72, wherein the poly(acrylamide) polymer is not a poly(N-isopropylacrylamide) polymer.

74. The article of any one of claim 72 or 73, wherein the poly(acrylamide) polymer comprises a poly poly(N,N-diethyl acrylamide) polymer.

75. The article of any one of claims 72-74, wherein the poly(acrylamide) polymer comprises a poly(N-ethylmethacrylamide) polymer.

76. The article of any one of claims 71-75, wherein the polymer comprises a poly(caprolactam).

77. The article of claim 76, wherein the poly(caprolactam) comprises poly(N-vinyl caprolactam).

78. The article of any one of claims 71-77, wherein the polymer comprises a poly(vinyl ether).

79. The article of claim 78, wherein the poly(vinyl ether) comprises a poly(methyl vinyl ether).

80. The article of any one of claim 78 or 79, wherein the poly(vinyl ether) comprises a poly(2-ethoxyethyl vinyl ether).

81. The article of any one of claims 71-80, wherein the polymer is a homopolymer.

82. The article of any one of claims 71-81, wherein the polymer is a block copolymer.

83. The article of any one of claims 71-82, wherein the polymer is a branched polymer.

84. The article of any one of claims 71-83, wherein the polymer is dissolved in the solution at a concentration of between 0.25 g/dL to 1 g/dL.

85. The article of any one of claims 71-84, wherein the charged lithium sodium magnesium silicate particle comprises Laponite.

86. The article of any one of claims 71-85, wherein the charged lithium sodium magnesium silicate particle is negatively charged.

87. The article of any one of claims 71-86, wherein the charged lithium sodium magnesium silicate particle is positively charged.

88. The article of any one of claims 71-87, wherein the charged lithium sodium magnesium silicate particle comprises a coating.

89. The article of any one of claims 71-88, wherein the charged lithium sodium magnesium silicate particle is present in the solution at a concentration of between 0.05 g/dL and 1 g/dL.

90. The article of any one of claims 71-89, wherein the charged lithium sodium magnesium silicate particles degrade into nontoxic components.

91. The article of any one of claims 71-90, wherein the solution further comprises one or more antibiotics.

92. The article of any one of claims 71-91, wherein the solution further comprises one or more clotting factors.

93. The article of claim 92, wherein the one or more clotting factors comprises thrombin.

94. The article of any one of claim 92 or 93, wherein the one or more clotting factors comprises clotting factor XIII.

95. The article of any one of claims 92-94, wherein the one or more clotting factors comprises clotting factor VII.

96. The article of any one of claims 71-95, wherein the solution further comprises one or more antifibrinolytic agents.

97. The article of claim 96, wherein the one or more antifibrinolytic agents comprises tranexamic acid.

98. The article of any one of claim 96 or 97, wherein the one or more antifibrinolytic agents comprises epsilon-aminocaproic acid.

99. The article of any one of claims 71-98, wherein the article is a hemostatic agent.

100. An article, comprising:
a solution comprising poly(N-vinyl caprolactam) and charged lithium sodium magnesium silicate particles.

101. A method, comprising:
administering, to a site of hemorrhage in a subject, a solution comprising a polymer and charged silicate nanoparticles; and
heating the solution at the site of hemorrhage to the subject's temperature to produce a hydrogel comprising the polymer and clotting proteins inter-dispersed with the charged silicate nanoparticles.

102. The method of claim 101, wherein the charged silicate nanoparticle binds one or more clotting proteins.

103. The method of claim 102, wherein binding one or more clotting proteins to the charged silicate nanoparticles concentrates the clotting factors within the site of hemorrhage.

104. The method of claim 101-103, wherein the hydrogel further comprises a blood clot.

105. The method of claim 104, wherein the blood clot has a maximum amplitude of between 55 mm to 80 mm.

106. The method of any one of claim 104 or 105, wherein the blood clot has an K value of between 1 min and 4 mins.

107. The method of any one of claims 104-106, wherein the blood clot has an R-value of between 4 min and 8 min.

108. The method of any one of claims 104-107, wherein the blood clot has an alpha-angle of between 47 degrees and 74 degrees.

109. The method of any one of claims 104-108, wherein the blood clot has an LY30% value of between 0% and 8% of the total mass of the blood clot.

110. The method of any one of claims 101-109, wherein heating the solution to produce the hydrogel uses the subjects body temperature.

111. The method of any one of claims 101-110, wherein heating the solution to produce the hydrogel uses an external heat source.

112. The method of any one of claims 101-111, wherein heating the solution causes the polymer to undergo a phase change from a liquid to a solid.
